SEQUENCE LISTING (45 sequences)

SEQ ID NO: 1 - protein, 188 aa, Woodchuck hepatitis virus
  VARIANT 61: Xaa = Cys or Ser
MDIDPYKEFGSSYQLLNFLPLDFFPDLNALVDTATALYEEELTGREHCSPHHTAIRQALVXWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 2 - protein, 149 aa, Woodchuck hepatitis virus
  VARIANT 61: Xaa = Cys or Ser
MDIDPYKEFGSSYQLLNFLPLDFFPDLNALVDTATALYEEELTGREHCSPHHTAIRQALVXWDELTKLIAWMSSNITSEQVRTIIVNHVNDTWGLKVRQSLWFHLSCLTFGQHTVQEFLVSFGVWIRTPAPYRPPNAPILSTLPEHTVI

SEQ ID NO: 3 - protein, 183 aa, Human hepatitis B virus
  VARIANT 61: Xaa = Cys or Ser
MDIDPYKEFGATVELLSFLPSDFFPSVRDLLDTASALYREALESPEHCSPHHTALRQAILXWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 4 - protein, 149 aa, Human hepatitis B virus
  VARIANT 61: Xaa = Cys or Ser
MDIDPYKEFGATVELLSFLPSDFFPSVRDLLDTASALYREALESPEHCSPHHTALRQAILXWGELMTLATWVGVNLEDPASRDLVVSYVNTNMGLKFRQLLWFHISCLTFGRETVIEYLVSFGVWIRTPPAYRPPNAPILSTLPETTVV

SEQ ID NO: 5 - protein, 188 aa, Artificial Sequence
  Synthetic Construct
  VARIANT 11, 12, 13, 14, 17, 21, 26, 27, 28, 29, 31, 35, 39, 41,43, 44, 45, 55, 59, 60, 61, 63, 66, 67, 69, 70, 72, 73, 74,76, 77, 78, 79, 80, 81, 83, 84, 85, 87, 88, 91, 92, 93,97, 100, 105, 112, 113, 116, 118, 130, 131, 146, 149, 154: Xaa = Any Amino Acid
  VARIANT 155, 156, 157, 158, 184, 185, 186, 187: Xaa = Any Amino Acid
MDIDPYKEFGXXXXLLXFLPXDFFPXXXXLXDTAXALYXEXLXXXEHCSPHHTAXRQAXXXWXELXXLXXWXXXNXXXXXXRXXXVXXVNXXXGLKXRQXLWFHXSCLTFGXXTVXEXLVSFGVWIRTPXXYRPPNAPILSTLPEXTVXRRRGXXXXXRSPRRRTPSPRRRRSQSPRRRRSQSXXXXC

SEQ ID NO: 6 - protein, 149 aa, Artificial Sequence
  Synthetic Construct
  VARIANT 11, 12, 13, 14, 17, 21, 26, 27, 28, 29, 31, 35, 39, 41,43, 44, 45, 55, 59, 60, 61, 63, 66, 67, 69, 70, 72, 73, 74,76, 77, 78, 79, 80, 81, 83, 84, 85, 87, 88, 91, 92, 93,97, 100, 105, 112, 113, 116, 118, 130, 131, 146, 149: Xaa = Any Amino Acid
MDIDPYKEFGXXXXLLXFLPXDFFPXXXXLXDTAXALYXEXLXXXEHCSPHHTAXRQAXXXWXELXXLXXWXXXNXXXXXXRXXXVXXVNXXXGLKXRQXLWFHXSCLTFGXXTVXEXLVSFGVWIRTPXXYRPPNAPILSTLPEXTVX

SEQ ID NO: 7 - protein, 108 aa, Human hepatitis B virus
MGQNLSTSNPLGFFPDHQLDPAFRANTANPDWDFNPNKDTWPDANKVGAGAFGLGFTPPHGGLLGWSPQAQGILQTLPANPPPASTNRQSGRQPTPLSPPLRNTHPQA

SEQ ID NO: 8 - protein, 55 aa, Human hepatitis B virus
MQWNSTTFHQTLQDPRVRGLYFPAGGSSSGTVNPVLTTASPLSSIFSRIGDPALN

SEQ ID NO: 9 - protein, 163 aa, Human hepatitis B virus
MGQNLSTSNPLGFFPDHQLDPAFRANTANPDWDFNPNKDTWPDANKVGAGAFGLGFTPPHGGLLGWSPQAQGILQTLPANPPPASTNRQSGRQPTPLSPPLRNTHPQAMQWNSTTFHQTLQDPRVRGLYFPAGGSSSGTVNPVLTTASPLSSIFSRIGDPALN

SEQ ID NO: 10 - protein, 226 aa, Human hepatitis B virus
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 11 - protein, 281 aa, Human hepatitis B virus
MQWNSTTFHQTLQDPRVRGLYFPAGGSSSGTVNPVLTTASPLSSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 12 - protein, 389 aa, Human hepatitis B virus
MGQNLSTSNPLGFFPDHQLDPAFRANTANPDWDFNPNKDTWPDANKVGAGAFGLGFTPPHGGLLGWSPQAQGILQTLPANPPPASTNRQSGRQPTPLSPPLRNTHPQAMQWNSTTFHQTLQDPRVRGLYFPAGGSSSGTVNPVLTTASPLSSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 13 - protein, 23 aa, Human hepatitis B virus
PAFRANTANPDWDFNPNKDTWPD

SEQ ID NO: 14 - protein, 25 aa, Human hepatitis B virus
LDPAFRANTANPDWDFNPNKDTWPD

SEQ ID NO: 15 - protein, 24 aa, Human hepatitis B virus
PASTNRQSGRQPTPLSPPLRNTHP

SEQ ID NO: 16 - protein, 21 aa, Human hepatitis B virus
MGQNLSTSNPLGFFPDHQLDP

SEQ ID NO: 17 - protein, 25 aa, Human hepatitis B virus
MGQNLSTSNPLGFFPDHQLDPAFRA

SEQ ID NO: 18 - protein, 17 aa, Human hepatitis B virus
PDHQLDPAFRANTANPD

SEQ ID NO: 19 - protein, 19 aa, Human hepatitis B virus
PDHQLDPAFRANTANPDWD

SEQ ID NO: 20 - protein, 33 aa, Human hepatitis B virus
MGQNLSTSNPLGFFPDHQLDPAFRANTANPDWD

SEQ ID NO: 21 - protein, 26 aa, Human hepatitis B virus
PDHQLDPAFRANTANPDWDFNPNKDT

SEQ ID NO: 22 - protein, 39 aa, Human hepatitis B virus
GQNLSTSNPLGFFPDHQLDPAFRAGGGGPASTNRQSGRQ

SEQ ID NO: 23 - protein, 36 aa, Human hepatitis B virus
MGQNLSTSNPLGFFPDHQLDPAFRANTANPDWDFNP

SEQ ID NO: 24 - protein, 49 aa, Human hepatitis B virus
MGQNLSTSNPLGFFPDHQLDPEEEEPASTNRQSGRQPTPLSPPLRNTHP

SEQ ID NO: 25 - protein, 15 aa, Human hepatitis B virus
DPRVRGLYFPAGGSS

SEQ ID NO: 26 - protein, 20 aa, Clostridium tetani
NNFTVSFWLRVPKVSASHLE

SEQ ID NO: 27 - protein, 19 aa, Artificial Sequence
  Synthetic Construct
GAGTGTGGATTCGCACTCC

SEQ ID NO: 28 - protein, 19 aa, Artificial Sequence
  Synthetic Construct
GAGGCGAGGGAGTTCTTCT

SEQ ID NO: 29 - protein, 58 aa, Artificial Sequence
  Synthetic Construct
  VARIANT 4, 5: Up to two can be present or absent
  VARIANT 6, 7, 8, 9, 10, 11, 12, 13, 14, 15, 16, 17, 18, 19, 20,21, 22, 23, 24, 25, 26, 27, 28, 29, 30, 31, 32, 33, 34, 35,36, 37, 38, 39, 40, 41, 42, 43, 44, 45, 46, 47, 48, 49,50, 51, 52, 53, 54, 55: Xaa = Any Amino Acid, and up to 49 can be present or absent
  VARIANT 56, 57: Up to two can be present or absent
GILEEXXXXXXXXXXXXXXXXXXXXXXXXXXXXXXXXXXXXXXXXXXXXXXXXXXEEL

SEQ ID NO: 30 - protein, 4 aa, Artificial Sequence
  Synthetic Construct
EEEE

SEQ ID NO: 31 - protein, 4 aa, Artificial Sequence
  Synthetic Construct
GGGG

SEQ ID NO: 32 - protein, 220 aa, Artificial Sequence
  Synthetic Construct
MDIDPYKEFGSSYQLLNFLPLDFFPDLNALVDTATALYEEELTGREHCSPHHTAIRQALVCWDELTKLIAWMSSNITSGILEEPASTNRQSGRQPTPLSPPLRNTHPEELEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 33 - protein, 213 aa, Artificial Sequence
  Synthetic Construct
MDIDPYKEFGSSYQLLNFLPLDFFPDLNALVDTATALYEEELTGREHCSPHHTAIRQALVCWDELTKLIAWMSSNITSGILMGQNLSTSNPLGFFPDHQLDPLEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 34 - protein, 214 aa, Artificial Sequence
  Synthetic Construct
MDIDPYKEFGSSYQLLNFLPLDFFPDLNALVDTATALYEEELTGREHCSPHHTAIRQALVCWDELTKLIAWMSSNITSPDHQLDPAFRANTANPDWDFNPNKDTEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 35 - protein, 250 aa, Artificial Sequence
  Synthetic Construct
MGPASTNRQSGRQPTPLSPPLRNTHPWLWGAMDIDPYKEFGSSYQLLNFLPLDFFPDLNALVDTATALYEEELTGREHCSPHHTAIRQALVSWDELTKLIAWMSSNITSGILEMGQNLSTSNPLGFFPDHQLDPAFRAELEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 36 - protein, 342 aa, Artificial Sequence
  Synthetic Construct
MDIDPNKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVCGGSGMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 37 - protein, 346 aa, Artificial Sequence
  Synthetic Construct
MDIDPNKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGGARASMDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 38 - protein, 5 aa, Artificial Sequence
  Synthetic Construct
CGGSG

SEQ ID NO: 39 - protein, 9 aa, Artificial Sequence
  Synthetic Construct
RRRGGARAS

SEQ ID NO: 40 - protein, 22 aa, Artificial Sequence
  Synthetic Construct
DIEYLNKIQNSLSTEWSPCSVT

SEQ ID NO: 41 - protein, 119 aa, Human hepatitis B virus
MGGWSSKPRQGMGTNLSVPNPLGFFPDHQLDPAFGANSNNPDWDFNPNKDHWPEAIKVGAGDFGPGFTPPHGGLLGWSPQAQGILTTVPAAPPPVSTNRQSGRQPTPISPPLRDSHPQA

SEQ ID NO: 42 - protein, 55 aa, Human hepatitis B virus
MQWNSTTFHQALLDPRVRGLYFPAGGSSSGTVNPVPTTVSPISSIFSRTGDPAPN

SEQ ID NO: 43 - protein, 174 aa, Human hepatitis B virus
MGGWSSKPRQGMGTNLSVPNPLGFFPDHQLDPAFGANSNNPDWDFNPNKDHWPEAIKVGAGDFGPGFTPPHGGLLGWSPQAQGILTTVPAAPPPVSTNRQSGRQPTPISPPLRDSHPQAMQWNSTTFHQALLDPRVRGLYFPAGGSSSGTVNPVPTTVSPISSIFSRTGDPAPN

SEQ ID NO: 44 - protein, 270 aa, Artificial Sequence
  Synthetic Construct
MDIDPYKEFGSSYQLLNFLPLDFFPDLNALVDTATALYEEELTGREHCSPHHTAIRQALVCWDELTKLIAWMSSEDPRVRGLYFPAGGSSEPSSEDPRVRGLYFPAGGSSEPSSEDPRVRGLYFPAGGSSEPSSNITSEQVRTIIVNHVNDTWGLKVRQSLWFHLSCLTFGQHTVQEFLVSFGVWIRTPAPYRPPNAPILSTLPEHTVIRRRGGARASRSPRRGTPSPRRRRSQSPRRRRSQSPSANCDIEYLNKIQNSLSTEWSPCSVT

SEQ ID NO: 45 - protein, 60 aa, Artificial Sequence
  Synthetic Construct
EDPRVRGLYFPAGGSSEPSSEDPRVRGLYFPAGGSSEPSSEDPRVRGLYFPAGGSSEPSS

(19) United States
(12) Patent Application Publication
(10) Pub. No.: US 2022/0411475 A1
(43) Pub. Date: Dec. 29, 2022

(54) HYBRID VIRUS-LIKE PARTICLES AND USE THEREOF AS A THERAPEUTIC HEPATITIS B VACCINE

(71) Applicant: VLP Biotech, Inc., San Diego, CA (US)

(72) Inventors: David R. MILICH, San Diego, CA (US); David C. WHITACRE, San Diego, CA (US)

(73) Assignee: VLP Biotech, Inc., San Diego, CA (US)

(21) Appl. No.: 17/777,589

(22) PCT Filed: Nov. 13, 2020
(86) PCT No.: PCT/US2020/060542

§ 371 Date: May 17, 2022

Related U.S. Application Data
(60) Provisional application No. 62/937,114, filed on Nov. 18, 2019.

(51) Int. Cl. C07K 14/02; A61K 39/12; C12N 15/74; C12N 15/79

(52) U.S. Cl. CPC C07K 14/02; A61K 39/12; C12N 15/74; C12N 15/79; A61K 2039/5258

(57) ABSTRACT

The present disclosure relates to hybrid hepadnavirus core antigens including one or more epitopes of a human hepatitis B vims (HBV) antigen. More specifically, the present disclosure relates to hybrid hepadnavirus core antigens in the form of fusion proteins containing a fragment of the PreS1 region of the HBV surface antigen inserted in a woodchuck hepadnavirus core antigen. The present disclosure further relates to hybrid hepadnavirus core antigens in the form of fusions proteins containing a truncated HBV core antigen and woodchuck hepadnavirus core antigen. Also provided are nucleic acids encoding the hybrid core antigens, and the use of the hybrid core antigens and nucleic acids for treating HBV-infected individuals.

### CROSS-REFERENCE TO RELATED APPLICATIONS

This application claims benefit of U.S. Provisional Application No. 62/937,114, filed Nov. 18, 2019, the contents of which are hereby incorporated by reference in their entirety.

### STATEMENT REGARDING FEDERALLY SPONSORED RESEARCH OR DEVELOPMENT

This invention was made with government support under Grant Nos. R01 AI049730 and R44 AI08819 awarded by The National Institutes of Health. The government has certain rights in the invention.

### SUBMISSION OF SEQUENCE LISTING AS ASCII TEXT FILE

The content of the following submission on ASCII text file is incorporated by reference in its entirety: a computer readable form (CRF) of the Sequence Listing (file name: 720222000440SEQLIST.TXT, date recorded: Nov. 13, 2020, size: 46 KB).

### FIELD

The present disclosure relates to hybrid hepadnavirus core antigens including one or more epitopes of a human hepatitis B virus (HBV) antigen. More specifically, the present disclosure relates to hybrid hepadnavirus core antigens in the form of fusion proteins containing a fragment of the PreS1 and/or PreS2 region of the HBV surface antigen inserted in a woodchuck hepadnavirus core antigen. The present disclosure further relates to hybrid hepadnavirus core antigens in the form of fusions proteins containing a truncated HBV core antigen and woodchuck hepadnavirus core antigen. Also provided are nucleic acids encoding the hybrid core antigens, and the use of the hybrid core antigens and nucleic acids for treating HBV-infected individuals.

### BACKGROUND

Most adults whom become infected with hepatitis B virus (HBV) recover completely and do not become chronically infected. In contrast, as many as 90% of infants and 25-50% of toddlers remain chronically HBV-infected (Fattovich et al., J Hepatol, 48:335-352, 2008). Vertical transmission of HBV is the major source of chronic infection in endemic areas, creating a cycle of perinatal infection, chronicity, and late term complications including fibrosis and hepatocellular carcinoma (HCC) with over 1 million deaths annually. The large number of HBV chronic carriers represents a significant health problem since they serve as a reservoir for further infection, as well as being at risk for the development of HCC. Therefore, in addition to worldwide vaccine programs to prevent new infections, methods for treating HBV chronic carriers are necessary to eradicate this hepatitis B disease.

Although a number of nucleotide and nucleoside analogs are quite effective at reducing HBV viral load, treatment is associated with poor sustained responses. Use of immunomodulatory pegylated-interferon alfa-2b, alone or in combination with antiviral drugs has resulted in improved, albeit still low rates of sustained response (Lok, N Engl J Med, 352:2743-2746, 2005). It was hoped that antiviral treatments would permit the immune system to “reset” allowing for recovery of innate/adaptive immunity. However, this approach has only been marginally successful. One consequence of the standard antiviral therapies is that reduced HBV replication and antigenic loads deprive the immune system of necessary antigenic stimuli.

Therapeutic vaccination was contemplated to compliment antiviral therapy by providing the necessary immunogenic stimulus to drive innate/adaptive immune responses. Despite the existence of safe and efficacious preventative vaccines for HBV, the preventative vaccines are not effective against chronic infection. The primary cause of chronic infection and the greatest impediment to developing a therapeutic vaccine is the direct and indirect effects of immune tolerance mediated primarily by the secreted hepatitis B e antigen (HBeAg) and the hepatitis B surface antigen (HBsAg) (Milich, Gastroenterology, 151:801-804, 2016; and Protzer et al., Gastroenterology, 151:801-804, 2016). The resulting defective cytotoxic T lymphocyte (CTL) responses, poor cytokine production, insufficient neutralizing antibody (nAb) levels and non-response to preventative HBsAg-based vaccination (a direct measure of immune tolerance) characterize chronic HBV infection. The current HBV treatments (nucleoside analogs and interferon-alpha) at best achieve a “functional cure” (reduced HBV DNA, normalized liver injury and anti-HBe seroconversion), but do not achieve a “complete cure” involving immune restoration and elimination of covalently-closed circular DNA (cccDNA) from all hepatocytes.

A number of therapeutic vaccine clinical trials have been conducted using the HBV envelope antigens (i.e., HBsAg, PreS2 and PreS1-containing subviral particles) singly or combined, delivered as proteins in adjuvant or as DNA constructs, all with rather disappointing results (Michel et al., J Hepatol, 54:1286-1296, 2011). In many of these studies antiviral drugs were also used in order to inhibit viral replication. Nevertheless, the HBV envelope vaccines to date have not demonstrated clear clinical efficacy. The lack of immunogenicity is clear from the inability of candidate vaccines to elicit neutralizing antibodies.

Thus, what is needed in the art antigenic compositions for eliciting or enhancing a HBsAg-reactive antibody response. Also needed are compositions for eliciting or enhancing a HBcAg-reactive T lymphocyte response. The use of compositions for eliciting or enhancing the above-mentioned humoral and cellular immune responses would solve a need in the art for a therapeutic vaccine regimen for preventing HBV spread within the liver and eradicating intracellular HBV in subjects chronically-infected with HBV.

### BRIEF SUMMARY

The present disclosure relates to hybrid hepadnavirus core antigens including one or more epitopes of a human hepatitis B virus (HBV) antigen. More specifically, the present disclosure relates to hybrid hepadnavirus core antigens in the form of fusion proteins containing a fragment of the PreS1 and/or PreS2 region of the HBV surface antigen inserted in a woodchuck hepadnavirus core antigen. The present disclosure further relates to hybrid hepadnavirus core antigens in the form of fusions proteins containing a truncated HBV core antigen and woodchuck hepadnavirus core antigen. Also provided are nucleic acids encoding the hybrid core antigens, and the use of the hybrid core antigens and nucleic acids for treating HBV-infected individuals.

### BRIEF DESCRIPTION OF THE DRAWINGS

FIG. 1 depicts cellular pathways for antigen presentation as a consequence of a therapeutic hepatitis B virus (HBV) vaccine regimen comprising administration of a virus-like particle (VLP) prime followed by a DNA boost to a HBV-infected individual. In particular, a woodchuck hepadnavirus core antigen (WHcAg) serves as a carrier of epitopes of the PreS1 region of the hepatitis B virus surface antigen (HBsAg), and epitopes of the hepatitis B virus core antigen (HBcAg) to elicit anti-PreS1 neutralizing antibodies and HBcAg-specific cytotoxic T lymphocytes (CTL).

FIG. 2A provides an alignment of amino acid sequences of woodchuck hepadnavirus core antigen (WHcAg) and hepatitis B virus core antigen (HBcAg), and a consensus amino acid sequence. Asterisks denote amino acid identity. The amino acid sequences are as follows: WHcAg sequence is set forth as SEQ ID NOW HBcAg is set forth as SEQ ID NO:3; and the consensus sequence is set forth as SEQ ID NO:5. In SEQ ID NO:1 and SEQ ID NO:3, X is cysteine or serine, while in SEQ ID NO:5, X can be any amino acid or absent, preferably wherein X is the amino acid residue of either of the aligned WHcAg or HBcAg sequences.

FIG. 2B provides an alignment of amino acid sequences of the PreS1 and PreS2 of HBV subtypes ayw (SEQ ID NO:9) and adr (SEQ ID NO:43), along with multiple PreS1 fragments (SEQ ID NOs:13-21) and a PreS2 fragment (SEQ ID NO:25).

FIG. 3A-B show that anti-PreS1-WHc VLP Abs bind multiple large/medium/small (L/M/S)-HBsAg particles of both major serotypes (ad and ay). Four L/M/S-HBsAg preparations, derived from HBV infected sera were used as solid-phase ligands to measure anti-PreS1 Ab binding by ELISA. Groups of 3 mice were immunized (IP) with 20 μg and boosted with 1.0 μg of the indicated PreS1-WHc VLPs and pooled sera were tested for binding to the panel of L/M/S-HBsAg antigens by ELISA. Endpoint titers (1/dilution) are shown. The Mab 18/7 was included for reference.

FIG. 4 illustrates the immunogenicity of PreS1-WHc VLP-1.6 in (wild type) WT and TLR7-knock-out (KO) Mice. Groups of 3 WT or TLR7-KO mice were immunized (IP) with a single dose of 20 μg of PreS1-WHc VLP-1.6 emulsified in incomplete Freund's adjuvant (IFA) and 3 and 5 weeks later IgG anti-PreS1 endpoint titers were determined on pooled sera by ELISA.

FIG. 5A and FIG. 5B show that immunization with PreS1-WHc VLP1.1 circumvents immune tolerance in HBe/HBcAg-transgenic (Tg) mice. Groups of 3 B10 WT, B10 HBeAg-Tg and B10 HBe/HBcAg double-Tg mice were immunized (IP) with a single 20 μg dose of HBcAg (FIG. 5A) or PreS1-WHc VLP1.1 (FIG. 5B) emulsified in IFA. Four weeks after immunization sera were collected, pooled and tested by ELISA for IgG anti-HBc, anti-WHc and anti-PreS1 Abs expressed as endpoint (1/dilution) titers.

FIG. 6A and FIG. 6B provides an analysis of CD4+ Th cell responses to PreS1-WHc VLP immunization in HBV-Tg mice. Groups of 3 HBV-Tg or \NIT (B6/BALBc) mice were immunized (SC) with 20 μg of either PreS1-WHc VLP-1.1+(FIG. 6A) or VLP-1.3 (FIG. 6B) emulsified in IFA. Four weeks later spleen cells were harvested and cultured (5×10⁵) with varying concentrations of the indicated WHcAg, HBcAg or WHc(W)- or HBc(H)-derived synthetic peptides. Culture supernatants were collected at 48 hrs for IL-2 determination and at 96 hrs for IFNγ determination by 2-site ELISA. The results represent single mice but are representative of 3 mice/group.

FIG. 7 shows neutralization of HBV infection by PreS1-WHc VLP antisera. Groups of 3 mice were immunized with the 6 depicted PreS1-WHc VLPs containing separate neutralizing B cell epitopes. Mice received a primary (20 μg) and a single booster (10 μg) immunization (IP) in IFA. Antisera were collected after the boost, pooled and the neutralization activity was determined in an in vitro infection assay using a modified hepatocyte cell line (HepaRG) infected with HDV particles coated with HBV envelope proteins (Blanchet and Sureau, J Virol, 80:11935-11945, 2006). The bottom panel represents a higher stringency neutralization assay.

FIG. 8A and FIG. 8B show that ti-PreS1 Abs prevent acute infection serum HBV from chronically-infected human liver chimeric mice. WT B10 mice were immunized and boosted with a mixture of PreS1-WHc VLPs-1.2, -1.3 and -1.6 (20 μg each) and 5 weeks after the boost sera was collected, pooled and used for the adoptive transfer. 0.2 ml of anti-PreS1-WHc sera or control anti-WHc sera were transferred into human liver chimeric mice prior to infection with 1×10⁶ GE copies of HBV/mouse in the control and acute groups. For the chronic group, 0.2 ml of anti-VLP sera was transferred 2 and 5 weeks after HBV infection. Serum HBV-DNA was measured at the indicated time points post-infection (FIG. 8A). Liver HBV-DNA was measured at termination (FIG. 8B).

FIG. 9A and FIG. 9B provide a comparison of delivery of PreS1-WHc VLP-1.6 as a DNA plasmid or as a protein. Groups of 5 WT B6 mice were either immunized (IM) with 50 μg of pVAX-VLP-1.6 DNA by electroporation (EP) using the Clinporator device (IGEA, Italy) and boosted one month later or were immunized (IM) with 20 μg of VLP-1.6 inn IFA and boosted with 10 μg one month later. FIG. 9A show the detection of IFNγ-specific spot forming cells (SFC)/10⁶ spleen cells as determined using a commercial ELISPOT assay. FIG. 9B shows Ab production after the primary (1°) and the boost (2°) as determined by ELISA. The MHC class I-restricted CD8+ CTL epitopes on WHcAg (W10-25) and on HBcAg (H93-100) for B6 mice are boxed, while the other peptides are MHC class II-restricted CD4+ Th cell epitopes.

FIG. 10A, FIG. 10B and FIG. 10C show production of hybrid WHcAg/HBcAg VLPs. Full-length WHcAg₁₈₈ and truncated HBcAg₁₄₉ genes were co-expressed in E. coli (FIG. 10A). The mixed dimer band was excised from the gel and run under partially-reducing conditions, fully reduced and run on a second gel (arrows). In another approach, the HBcAg₁₄₉ gene was fused to the WHcAg₁₈₈ gene with a dimer linker to form a “single-chain dimer”, which was expressed as a single open reading frame in E. coli. VLP-347 (FIG. 10B) includes the dimer linker CGGSG (SEQ ID NO:38), and VLP-372 includes the dimer linker RRRGGARAS (SEQ ID NO:39). Purified VLP-347 was analyzed in capture ELISAs specific for hybrid WHcAg/HBcAg VLPs and incapable of detecting homologous WHcAg or HBcAg VLPs. Purified VLP-372 was analyzed as a solid-phase antigen on an ELISA plate and was found to present both WHcAg and HBc/HBeAg epitopes, which were recognized by WHcAg and HBc/HBeAg-specific monoclonal antibodies.

FIG. 11 shows that hybrid WHcAg/HBcAg VLP DNA constructs can prime efficient HBcAg-specific CTL. Groups of 3 WT B6 mice were immunized (SC) with DNA constructs (100 μg, 2 doses) encoding VLP-347, WHcAg₁₈₈/HBcAg₁₄₉ or HBcAg alone. Splenic CTL or Th cell (5×10⁵) IFNγ responses recalled by a panel of peptides corresponding to CTL or Th epitopes and whole protein antigens are shown. HBcAg-specific CTL (†) and WHcAg-hetero-specific Th cell (‡) responses are highlighted. IFNγ was measured in 4 day culture supernatants by two-site ELISA. Data is from single mice and is representative of 3 mice/group.

FIG. 12A and FIG. 12B show that tetanus toxoid (TT) priming provides hetero-specific T cell help for VLPs carrying a TT epitope. Groups of 3 mice (B10 strain) were either first primed with 20 μg tetanus toxin fragment C (TTFc) in IFA to mimic TT immunization in humans, or were unprimed. Two months later TTFc-primed and unprimed mice were injected with 10 μg hybrid WHc-TT950-969 VLPs in saline. FIG. 12A shows anti-WHc antibody levels determined by ELISA from sera pooled at 2, 6 and 28 weeks. FIG. 12B shows splenic T cell recognition of the TT950-969 peptide as measured by harvesting spleen cells and culturing with the TT950-969 peptide, followed by IL-2 determination by ELISA.

FIG. 13. shows neutralization of HBV infection by PreS2-WHcAg VLP antisera. Groups of 3 mice were immunized with the “VRI010c” PreS2-WHcAg VLPs containing a neutralizing B cell epitope from the PreS2 region of HBsAg. Mice received a primary (20 μg) and a single booster (10 μg) immunization (IP) in IFA. Antisera were collected after the boost, pooled and the neutralization activity was determined in an in vitro infection assay using a modified hepatocyte cell line (HepaRG) infected with HDV particles coated with HBV envelope proteins.

### DETAILED DESCRIPTION

An effective prophylactic hepatitis B virus (HBV) vaccine has long been available but is ineffective for chronic infection. The primary cause of chronic hepatitis B (CHB) and greatest impediment for a therapeutic vaccine is the direct and indirect effects of immune tolerance to HBV antigens. The resulting defective CD4+/CD8+ T cell response, poor cytokine production, insufficient neutralizing antibody (nAb) and poor response to HBsAg vaccination characterize CHB infection. The present disclosure describes the development of virus-like-particles (VLPs) that elicit nAb to prevent viral spread and prime CD4+/CD8+ T cells to eradicate intracellular HBV. Neutralizing B cell epitopes from the envelope PreS1 region were consolidated onto a species-variant of the HBV core protein, the woodchuck hepatitis core antigen (WHcAg). PreS1-specific B cell epitopes were chosen because of preferential expression on HBV virions. Because WHcAg and HBcAg are not cross-reactive at the B cell level and only partially cross-reactive at the CD4+/CD8+ T cell level, CD4+ T cells specific for WHcAg-unique T cell sites can provide cognate T-B cell help for anti-PreS1 Ab production that is not curtailed by immune tolerance. Immunization of immune tolerant HBV transgenic (Tg) mice with PreS1-WHc VLPs elicited levels of high titer anti-PreS1 nAbs equivalent to wild type mice. Passive transfer of PreS1 nAbs into human-liver chimeric mice prevented acute infection and cleared serum HBV from mice previously infected with HBV in a model of CHB. At the T cell level, PreS1-WHc VLPs and hybrid WHcAg/HBcAg DNA immunogens elicited HBcAg-specific CD4+ Th and CD8+ CTL responses.

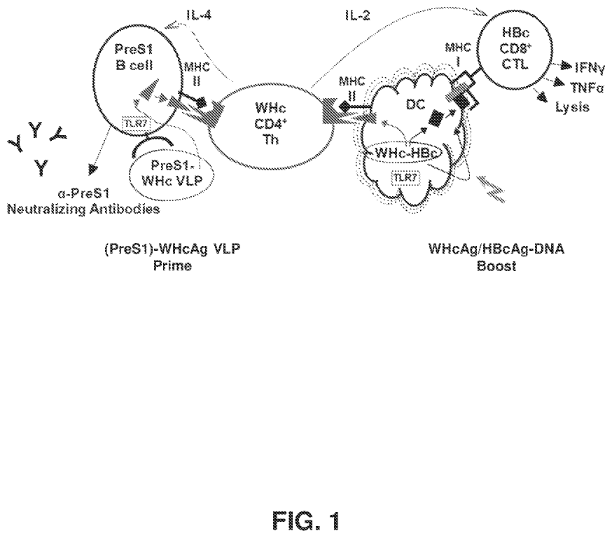

The relative scarcity of PreS1 antigen relative to the major HBsAg is a limiting factor for anti-PreS1 nAb production during a natural HBV infection. The capacity of the highly immunogenic WHcAg carrier to display multiple PreS1 neutralizing B cell epitopes overcomes this limitation. For example, 240 copies of each of the PreS1 B cell epitopes are displayed per PreS1-WHc VLP. A combined PreS1-WHcAg VLP (e.g., VLP-1.6 and/or VLP-1.9) vaccine formulated in an adjuvant suitable for human use given in a prime/boost protocol with an optimized WHcAg/HBcAg DNA construct is a strong candidate therapeutic HBV vaccine capable of circumventing immune tolerance and eliciting multiple PreS1 nAb specificities, as well as HBcAg-specific CD8+ CTL to target intracellular HBV DNA including cccDNA (see FIG. 1). Although a PreS1-WHc VLP prime-hybrid WHcAg/HBcAg DNA boost regimen could be given as a monotherapy, combination with an antiviral agent may enhance efficacy by reducing viral load. Inserting multiple neutralizing B cell PreS1 epitopes will mitigate the possibility of nAb escape mutants, which may be problematic when treating an established HBV infection. Bacterial production of PreS1-WHc VLPs together with a WHcAg/HBcAg DNA immunogen would be cost efficient and compatible with any antiviral treatment for maximum efficacy.

The present disclosure provides antigenic compositions comprising a hybrid hepadnavirus core antigen, wherein the hybrid core antigen is a fusion protein comprising a first portion of a human hepatitis B virus surface antigen (HBsAg), and a woodchuck hepadnavirus core antigen (WHcAg), wherein the first portion of the HBsAg comprises 8 to 50 amino acids of one or both of the PreS1 domain and the PreS2 domain of the human hepatitis B virus (HBV) large surface antigen, and wherein the fusion protein is capable of assembling as a hybrid VLP.

In addition to use as a therapeutic HBV vaccine, other possible applications for PreS1-WHc VLPs include: Use as a preventative vaccine in low-to-nonresponders to the conventional HBsAg vaccine; vaccination of pregnant HBV+ carrier mothers in order to provide passive transfer of PreS1-specific neutralizing Abs to block transmission during and after birth; an immunotherapy for chronic HDV infection; and prior to immunosuppressive therapy, vaccination of HBV+ liver transplant recipients in order to prevent infection of the new liver.

### Woodchuck Hepadnavirus Core Antigen

The woodchuck hepadnavirus core antigen (WHcAg) was chosen as a carrier in part because it is a multimeric, self-assembling, virus-like particles (VLP). The basic subunit of the core particle is a 21 kDa polypeptide monomer that spontaneously assembles into a 240 subunit particulate structure of about 34 nm in diameter. The tertiary and quaternary structures of hepadnavirus core particles have been elucidated (Conway et al., Nature, 386:91-94, 1997). The immunodominant B cell epitope on hepadnavirus core particles is localized around amino acids 76-82 (Schodel et al., J Exp Med, 180:1037-1046, 1994) forming a loop connecting adjacent alpha-helices. This observation is consistent with the finding that a heterologous antigen inserted within the 76-82 loop region of HBcAg was significantly more antigenic and immunogenic than the antigen inserted at the N- or C-termini and, importantly, more immunogenic than the antigen in the context of its native protein (Schodel et al., J Virol, 66:106-114, 1992).

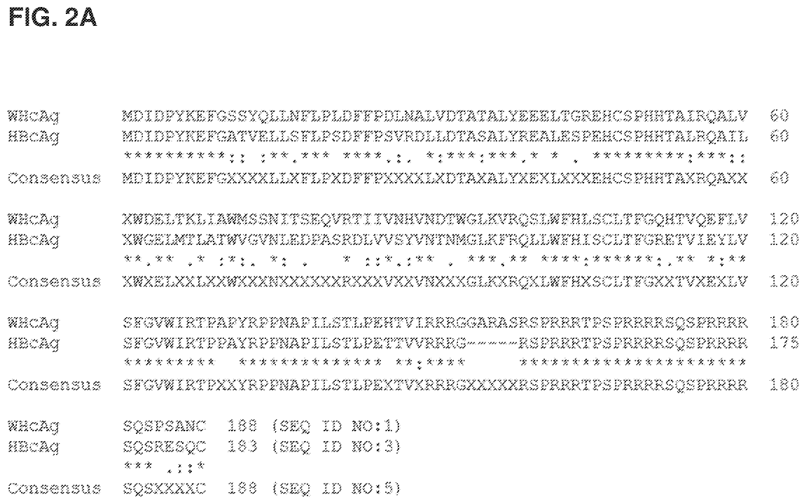

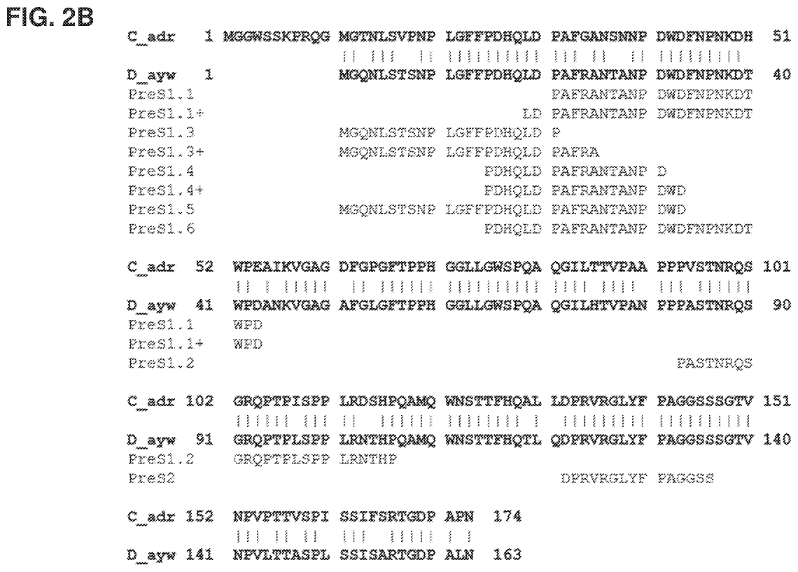

Full length and truncated wild type WHcAg cores, as well as recombinant WHcAg cores containing various mutations are suitable for use as fusion partners with Pre-S1 HBsAg and/or HBcAg, or fragments thereof, for production of hybrid VLPs. A preferred WHcAg is a full length WHcAg comprising the amino acid sequence of SEQ ID NO:1 or the sequence at least 95% (e.g., at least 95%, 96%, 97%, 98%, or 99%) identical thereto. In some embodiments, the WHcAg is a variant including from 1 to 9 amino acid differences with respect to the amino acid sequence of SEQ ID NO:1. That is, the WHcAg variant may include 1, 2, 3, 4, 5, 6, 7, 8 or 9 differences with respect to SEQ ID NO:1. In some embodiments, the differences include one or more of an insertion, a deletion, a substitution or combinations thereof. In some embodiments, the WHcAg comprises the consensus sequence of SEQ ID NO:5 (see, FIG. 2). In some embodiments, the differences include substitution of at least one X residue in the consensus sequence of SEQ ID NO:5 with the corresponding residue(s) of a representative HBcAg consisting of the amino acid sequence of SEQ ID NO:3 (e.g., A130P and P131A substitutions in WHcAg). In some embodiments, the differences include a conservative substitution of at least one residue in SEQ ID NO:1. In other embodiments, the differences include a non-conservative substitution of at least one residue in SEQ ID NO:1 (e.g., C61S substitution of WHcAg).

As described in more detail below and in Example 1, the PreS1-WHcAg VLPs were designed to include at least one PreS1 B cell epitope within the WHcAg immunodominant loop extending from residues 76-82 (Δ1 mutation) and/or at the N-terminus of the WHcAg. The WHcAg may be altered to reduce endogenous WHcAg-specific B cell epitopes in order to reduce WHcAg-specific antigenicity and/or immunogenicity without negatively affecting the antigenicity and/or immunogenicity of PreS1 B cell epitopes inserted within the WHcAg. The mutations designed to decrease WHcAg-specific antigenicity and/or immunogenicity are designated as Δ2-Δ7 mutations or modifications. Details of the modified WHcAg carrier platforms for presentation of heterologous antigens (hAg) such as HBV-PreS1 are known in the art (see, e.g., U.S. Pat. No. 10,300,124 of VLP Biotech, Inc., especially Table III and FIG. 1A, which are hereby incorporated by reference).

### Hepatitis B Virus Surface Antigen

As described herein, the hybrid VLPs of the present disclosure comprise a first portion of a human hepatitis B virus surface antigen (HBsAg) comprising 8 to 50 amino acids of one or both of the PreS1 domain and the PreS2 domain of the human hepatitis B virus (HBV) surface antigen (HBsAg). In some embodiments, the portion of the HBsAg is inserted at the N-terminus or an internal position of the WHcAg selected from the group consisting of 61, 71, 72, 73, 74, 75, 76, 77, 78, 81, 82, 83, 84, 85 and 92 as numbered according to SEQ ID NO:1

In some embodiments, PreS1-specific B cell epitopes of the HBsAg were chosen because of preferential expression on HBV virions. Accordingly, at least one portion of the PreS1 domain of the large HBsAg is inserted in the WHcAg to form a fusion protein capable of assembling as a hybrid PreS1-WHcAg virus-like particle (VLP). A preferred portion of the HBsAg comprises at least 8 amino acids, preferably from 8 to 50 amino acids of the PreS1 domain. In some embodiments, the amino acid sequence of the PreS1 domain is at least 95% identical to SEQ ID NO:7 or SEQ ID NO:41. In some embodiments, the at least one portion of the PreS1 domain comprises one of the group consisting of SEQ ID NOs:13-24.

In further embodiments, at least one portion of the PreS2 domain of the large HBsAg is inserted in the WHcAg to form a fusion protein capable of assembling as a hybrid PreS2-WHcAg VLP. A preferred portion of the HBsAg comprises at least 8 amino acids, preferably from 8 to 50 amino acids of the PreS2 domain. In some embodiments, the amino acid sequence of PreS2 domain is at least 95% identical to SEQ ID NO:8 or SEQ ID NO:42. In some embodiments, the at least one portion of the PreS2 domain comprises SEQ ID NO:25 or SEQ ID NO:45.

In some embodiments, the portion of the HBsAg (i.e., PreS1 or PreS2 fragment) comprises one B cell epitope, while in others it comprises two, three, four or five B cell epitopes, or even a larger plurality of B cell epitopes. In some embodiments, the portion of the HBsAg further comprises one T cell epitope, or it comprises two, three, four or five T cell epitopes, or even a larger plurality of T cell epitopes. In some embodiments, the T cell epitope is a helper T (Th) cell epitope (MHC class II-restricted epitope). In some embodiments, the T cell epitope is a cytotoxic T cell (CTL) epitope (MHC class I-restricted epitope).

The amino acid sequences of exemplary portions of the HBsAg are listed in Table I. Details of exemplary hybrid PreS1-WHcAg VLPs are provided in Table II.

TABLE 1
Sequences of HBV PreS1 and PreS2 Fragments

| | SEQ | | | |
|---|---|---|---|---|
| | ID | ayw | adw | |
| Epitope | NO: | aa # | aa # | Sequence |
| PreS1.1 | 13 | 21-43 | 32-54 | PAFRANTANPDWDFNPNKDTWPD |
| PreS1.1+ | 14 | 19-43 | 30-54 | LDPAFRANTANPDWDFNPNKDTW |
| | | | | PD |
| PreS1.2 | 15 | 83-106 | 94-117 | PASTNRQSGRQPTPLSPPLRNTH |
| | | | | P |
| PreS1.3 | 16 | 1-21 | 12-32 | MGQNLSTSNPLGFFPDHQLDP |
| PreS1.3+ | 17 | 1-25 | 12-36 | MGQNLSTSNPLGFFPDHQLDPAF |
| | | | | RA |
| PreS1.4 | 18 | 15-31 | 26-42 | PDHQLDPAFRANTANPD |
| PreS1.4+ | 19 | 15-33 | 26-44 | PDHQLDPAFRANTANPDWD |
| PreS1.5 | 20 | 1-33 | 12-44 | MGQNLSTSNPLGFFPDHQLDPAF |
| | | | | RANTANPDWD |
| PreS1.6 | 21 | 15-40 | 26-51 | PDHQLDPAFRANTANPDWDFNPN |
| | | | | KDT |
| PreS1.7 | 22 | 2-25, | 13-36, | GQNLSTSNPLGFFPDHQLDPAFR |
| | | 83-93 | 94-104 | A GGGGPASTNRQSGRQ |
| PreS1.8 | 23 | 1-36 | 12-47 | MGQNLSTSNPLGFFPDHQLDPA |
| | | | | FRANTANPDWDFNP |
| PreS1.9 | 24 | 1-21 | 12-32, | MGQNLSTSNPLGFFPDHQLDP |
| | | 83-106 | 94-117 | EEEEPASTNRQSGRQPTPLSP |
| | | | | PLRNTHP |
| PteS2 | 25 | 122-136 | 133-147 | DPRVRGLYFPAGGSS |

TABLE II
Details of Hybrid PreS1-WHcAg VLPs^{∧}

| | | N- | Linker | Loop | Linker |
|---|---|---|---|---|---|
| VLP No | VLP Name | insert | N-side | Insert | C-side |
| VRI010a | FLw2-HBV-PreS1.1-78 | — | GIL | 21-43 | L |
| VRI010b | FLw2-HBV-PreS1.2-78 | — | GILEE | 83-106 | EEL |
| VRI034 | FLw-HBV-PreS1.3-78 | — | GIL | 1-21 | L |
| VRI035 | FLw-HBV-PreS1.4-78 | — | GIL | 15-31 | L |
| VLP035 | FLw2-HBVPreS1.1(+)-78 | — | GIL | 19-43 | L |
| VLP036 | FLw2-HBV-PreS1.3(+)-78 | — | GIL | 1-25 | L |
| VLP037 | FLw2-HBV-PreS1.4(+)-78 | — | GIL | 15-33 | L |
| VLP038 | FLw2-HBV-PreS1.5-78 | — | GIL | 1-33 | L |
| VLP039 | FLw2-HBV-PreS1.3(+)E-78 | — | GILEE | 1-25 | EL |
| VLP054 | FLw2-HBV-PreS1.1-78/79* | — | fuse | 21-43 | fuse |
| VLP055 | FLw2-HBV-Pres 1.1(+)-78/79* | — | fuse | 19-43 | fuse |
| VLP056 | FLw2-HBV-PreS1.4-78/79* | — | fuse | 15-31 | fuse |
| VLP057 | FLw2-HBV-PreS1.4(+)-78/79* | — | fuse | 15-33 | fuse |
| VLP100 | FLw2-HBV-PreS1.4(+)-78N* | — | — | 15-33 | L |
| VLP101 | FLw2-HBV-PreS1.6-78/79* | — | fuse | 15-40 | fuse |
| VLP102 | FLw2-HBV-PreS1.7-78/79* | — | fuse | 2-25(GGGG) | fuse |
| | | | | 83-93 | |
| VLP103 | FLw-HBV-(NtA-PreS1.3+)- | 1-25 | GILEE | 83-106 | EEL |
| | PreS1.2-78 | | | | |
| VLP104 | FLw2-HBV-(NtA-PreS1.2)- | 83-106 | — | 15-33 | L |
| | PreS1.4(+)-78N* | | | | |
| VLP105 | FLw-HBV(NtA-PreS1.3+) | 1-25 | fuse | 19-43 | fuse |
| | PreS1.1(+)-78/79* | | | | |
| VLP106 | FLw-HBV-(NtA-PreS1.3+)- | 1-25 | fuse | 21-43 | fuse |
| | PreS1.1-78/79* | | | | |
| VLP115 | FLw2-HBV-(NtA-PreS1.2) | 83-106 | — | none | — |
| VLP116 | FLw-HBV-(NtA-PreS1.3+) | 1-25 | — | none | — |
| VLP152 | FLw2-HBV- (NtA-PreS1.2)- | 83-106 | GIL | 1-21 | L |
| | PreS1.3-78 | | | | |
| VLP153 | FLw2(C61S)-HBV-PreS1.3(+)E-78 | — | GIL | 1-25 | L |
| VLP154 | FLw2(C61S)-HBV-PreS1.6-78/79* | — | fuse | 15-40 | fuse |
| VLP157 | FLw2(C61S)-HBV-(NtA-PreS1.2)- | 83-106 | GIL | 1-21 | L |
| | PreS1.3-78 | | | | |
| VLP158 | FLw2(C61S)-HBV-(NtA-PreS1.2)- | 83-106 | GIL | 1-25 | L |
| | PreS13(+)E-78 | | | | |
| VLP191 | FLw2-HBV-PreS1.6-78C* | — | GIL | 15-40 | fuse |
| VLP192 | FLw2-HBV-PreSL6-78N* | — | fuse | 15-40 | L |
| VLP193 | FLw2-HBV-PreS1.6-78 | — | GIL | 15-40 | L |
| VLP194 | FLw2-HBV-PreS1.8-78 | — | GIL | 1-36 | E |
| VLP195 | FLw2(C61S)-HBV-PreS1.8-78 | — | GIL | 1-36 | L |
| VLP207 | FLw2-HBV-PreSL9E-78 | — | GILE | 1-21 EEEE | EL |
| | | | | 83-106 | |
| VLP208 | FLw2(C61S)-HBV-PreS1.9E-78 | — | GILE | 1-21 EEEE | EL |
| | | | | 83-106 | |

^{∧}(C61S) indicates the WHcAg has a serine a position 61. Inserts comprise sequences of the ayw subtype. Loop inserts, if made, were inserted at position 78.

In some instances, in which a PreS1 sequence is inserted at the N-terminus of the WHcAg, the PreS1 sequence is inserted after the methionine of position 1 of the WHcAg. In other instances in which the PreS11 sequence begins with a methionine, the PreS1 sequence replaces the methionine of position 1 of the WHcAg (i.e., the VLP sequence begins with a single methionine). In some instances in which the PreS1 sequence is inserted in an internal position of the WHcAg it is fused in frame without a linker (e.g., between positions 78 and 79 of the WHcAg). In other instance in which the PreS1 sequence is inserted in an internal position of the WHcAg it is inserted as a linker/insert combination according to the formula GIL(E)y-Xn-(E)zL (SEQ ID NO:29, in which both y and z are in integers independently selected from the group consisting of 0, 1, and 2, and wherein Xn is the PreS1 sequence).

As indicated above, a preferred portion of the HBsAg consists of from 8 to 50 amino acids of the PreS1 domain (PreS1 fragment). In other embodiments, the PreS1 fragment is 10 to 50 amino acids in length, preferably 15 to 45 amino acids in length, or preferably 20 to 40 amino acids in length. In some embodiments, the length PreS1 fragment is within any range having a lower limit of 8, 9, 10, 11, 12, 13, 14, 15, 16, 17, 18, 19 or 20 amino acids and an independently selected upper limit of 50, 45, 40, 35, 30, 25 or 20 amino acids in length, provided that the lower limit is less than the upper limit.

### Hepatitis B Core Antigen

Complete recovery from acute HBV infection requires cellular immune responses, especially multi-specific, polyclonal CTL responses. Further, patients with chronic HBV who experience remission have demonstrable CTL and Th responses. In contrast, the CTL and Th responses are undetectable or very weak in patients with ongoing chronic infection. Therefore, DNA constructs were designed to circumvent immune tolerance at the level of T cell help for CTL by taking advantage of the same technology used to bypass poor T cell help for production of anti-PreS1 antibodies. Co-expression of the HBcAg linked with the WHcAg in DNA vectors allows the foreign Th epitopes on WHcAg to elicit “hetero-specific” T cell help for HBcAg-specific CTL, which can be “helpless” in the context of chronic HBV infection. In particular, absent or defective (i.e., PD-1+) HBcAg-specific (“homo-specific”) Th cells can be replaced with WHcAg-specific (“hetero-specific”) Th cells. However, the WHcAg-specific Th cell epitopes must be physically linked to the HBcAg-specific CTL epitopes within the same VLP in order to ensure that both are taken up by the same dendritic cell (DC) or other antigen presenting cell as illustrated in FIG. 1. T cell help for CTL function is not mediated directly by Th-CTL interaction as exists in the direct Th-B cell interaction. T cell help for CTLs is mediated indirectly through activation of DCs or other antigen presenting cell.

Thus in some embodiments the present disclosure provides polynucleotides, expression constructs and vector encoding a fusion protein comprising a human hepatitis B virus core antigen (HBcAg) and a woodchuck hepadnavirus core antigen (WHcAg), wherein the fusion protein is capable of assembling as a hybrid HBcAg-WHcAg virus-like particle (VLP). In some embodiments, the HBcAg is truncated at residue 149 or 150. In some embodiments, the WHcAg is full length. In some embodiments, the amino acid sequence of the HBcAg is at least 95% identical to SEQ ID NO:4. In some embodiments, the amino acid sequence of the WHcAg is at least 95% identical to SEQ ID NO:1. In further embodiments, a dimer linker of from 5-15 amino acids in length is inserted between the amino acid sequence of the HBcAg and the amino acid sequence of the WHcAg, optionally wherein the dimer linker comprises the amino acid sequence of SEQ ID NO:38 or SEQ ID NO:39. In exemplary embodiments, the amino acid sequence of the hybrid HBcAg-WHcAg virus-like particle (VLP) is at least 95% identical to SEQ ID NO:36 or SEQ ID NO:37.

### Antigenic and Immunogenic Characterization of Hybrid, WHcAg-hAg VLPs

Determination as to whether a given heterologous antigen (e.g., PreS1 fragment of the large HBsAg or HBcAg) of a hybrid core antigen comprises a B cell epitope can be made by analyzing heterologous antigen-specific antibody-binding of serum of a subject immunized with the hybrid core antigen (or polynucleotide encoding the hybrid core antigen). Determination as to whether a given heterologous antigen of a hybrid core antigen comprises a Th cell epitope can be made by analyzing heterologous antigen-induced proliferation or cytokine secretion by peripheral blood lymphocytes (PBL) of a subject immunized with the hybrid core antigen (or polynucleotide encoding the hybrid core antigen). Determination as to whether a given heterologous antigen of a hybrid core antigen comprises a CTL cell epitope can be made by analyzing heterologous antigen-specific lysis of a target cell that expresses the heterologous antigen by CTL expanded from PBL of a subject immunized with a polynucleotide encoding the hybrid core antigen. Other methods of determining whether a heterologous antigen or fragment thereof comprises B, Th and/or CTL epitopes are known in the art.

### Antigenicity

Prior to immunogenicity testing, hybrid WHcAg-hAg VLPs are characterized for expression, particle assembly, and ability to bind a hAg-specific antibody. The same capture ELISA system used to detect hybrid VLPs in bacterial lysates may be used for purified particles. In brief, expression, particle assembly, and antibody binding are assayed by ELISA. SDS-PAGE and Western blotting are used to assess the size and antigenicity of hybrid VLPs.

### Immunogenicity

The immune response to hybrid VLPs is assessed. In addition to anti-insert, anti-hAg-protein and anti-WHcAg antibody endpoint titers, antibody specificity, isotype distribution, antibody persistence and antibody avidity are monitored. Immune sera are compared to the activity of a reference antibody by ELISA and neutralization assays. Immune responses are tested in vivo in various mammalian species (e.g., rodents such as rats and mice, nonhuman primates, humans, etc.).

### Compositions

The compositions of the present disclosure comprise a hybrid woodchuck hepadnavirus core antigen or a polynucleotide encoding the hybrid core antigen, wherein the hybrid core antigen is a fusion protein comprising a heterologous polypeptide and a woodchuck hepadnavirus core antigen, wherein the fusion protein is capable of assembling as a hybrid virus-like particle (VLP). In some embodiments, the heterologous polypeptide comprises at least one B cell epitope (e.g., capable of being bound by an antibody). In preferred embodiments, the composition is an antigenic composition. In some embodiments, the composition further comprises a pharmaceutically acceptable excipient, diluent, adjuvant, or combinations thereof.

Exemplary “diluents” include sterile liquids such as sterile water, saline solutions, and buffers (e.g., phosphate, tris, borate, succinate, histidine, etc.). Exemplary “excipients” are inert substances include but are not limited to polymers (e.g., polyethylene glycol), carbohydrates (e.g., starch, glucose, lactose, sucrose, cellulose, etc.), and alcohols (e.g., glycerol, sorbitol, xylitol, etc.).

Adjuvants are broadly separated into two classes based upon their primary mechanism of action: vaccine delivery systems (e.g., emulsions, microparticles, iscoms, liposomes, etc.) that target associated antigens to antigen presenting cells; and immunostimulatory adjuvants (e.g., LPS, MLP, CpG, etc.) that directly activate innate immune responses.

### Traditional and Molecular Adjuvants

Although adjuvants are not required when using the WHcAg delivery system, some embodiments of the present disclosure employ traditional and/or molecular adjuvants. Specifically, immunization in saline effectively elicits anti-insert antibody production. However, formulation in non-inflammatory agents such as IFA (mineral oil), Montanide ISA 720 (squalene), and aluminum phosphate (AIP04), enhance immunogenicity. Additionally, administration of WHcAg results in the production of all four IgG isotypes, regardless of which if any adjuvant is employed. Inclusion of a CpG motif also enhances the primary response. Moreover, use of an inflammatory adjuvant such as the Ribi formulation is not more beneficial than is the use of non-inflammatory adjuvants, indicating that the benefits of the adjuvants result from a depot effect rather than from non-specific inflammation. Thus, the core platform is used with no adjuvant or with non-inflammatory adjuvants depending upon the application and the quantity of antibody desired. In some embodiments of the present disclosure, IFA is used in murine studies, whereas alum or squalene is used in human studies. In instances where it is desirable to deliver hybrid WHcAg particles in a single dose in saline, a molecular adjuvant is employed. A number of molecular adjuvants are employed to bridge the gap between innate and adaptive immunity by providing a co-stimulus to target B cells or other APCs.

### Other Molecular Adjuvants

Genes encoding the murine CD40L (both 655 and 470 nucleic acid versions) have been used successfully to express these ligands at the C-terminus of WHcAg (See, WO 2005/011571). Moreover, immunization of mice with hybrid WHcAg-CD40L particles results in the production of higher anti-core antibody titers than does the immunization of mice with WHcAg particles. However, lower than desirable yields of purified particles have been obtained. Therefore, mosaic particles containing less than 100% CD40L-fused polypeptides are produced to overcome this problem. The other molecular adjuvants inserted within the WHcAg, including the C3d fragment, BAFF and LAG-3, have a tendency to become internalized when inserted at the C-terminus. Therefore tandem repeats of molecular adjuvants are used to resist internalization. Alternatively, various mutations within the so-called hinge region of WHcAg, between the assembly domain and the DNA/RNA-binding region of the core particle are made to prevent internalization of C-terminal sequences. However, internalization represents a problem for those molecular adjuvants such as CD40L, C3d, BAFF and LAG-3, which function at the APC/B cell membrane. In contrast, internalization of molecular adjuvants such as CpG DN is not an issue as these types of adjuvants function at the level of cytosolic receptors.

Another type of molecular adjuvant or immune enhancer is the inclusion within hybrid core particles of a CD4+ T cell epitope, preferably a “universal” CD4+ T cell epitope that is recognized by a large proportion of CD4+ T cells (such as by more than 50%, preferably more than 60%, more preferably more than 70%, most preferably greater than 80%), of CD4+ T cells. In one embodiment, universal CD4+ T cell epitopes bind to a variety of human MHC class II molecules and are able to stimulate T helper cells. In another embodiment, universal CD4+ T cell epitopes are preferably derived from antigens to which the human population is frequently exposed either by natural infection or vaccination (Falugi et al., Eur J Immunol, 31:3816-3824, 2001). A number of such universal CD4+ T cell epitopes have been described including, but not limited to: Tetanus Toxin (TT) residues 632-651; TT residues 950-969 (NNFTVSFWLRVPKVSASHLE set forth as SEQ ID NO:26); TT residues 947-967, TT residues 830-843, TT residues 1084-1099, TT residues 1174-1189 (Demotz et al., Eur J Immunol, 23:425-432, 1993); Diphtheria Toxin (DT) residues 271-290; DT residues 321-340; DT residues 331-350; DT residues 411-430; DT residues 351-370; DT residues 431-450 (Diethelm-Okita et al., J Infect Dis, 1818:1001-1009, 2000); Plasmodium falciparum circumsporozoite (CSP) residues 321-345 and CSP residues 378-395 (Hammer et al., Cell, 74:197-203, 1993); Hepatitis B antigen (HBsAg) residues 19-33 (Greenstein et al., J Immunol, 148:3970-3977, 1992); Influenza hemagglutinin residues 307-319; Influenza matrix residues 17-31 (Alexander et al., J Immunol, 164:1625-1633, 2000); and measles virus fusion protein (MVF) residues 288-302 (Dakappagari et al., J Immunol, 170:4242-4253, 2003).

### Methods of Inducing an Immune Response

The present disclosure provides methods for eliciting an immune response in an animal in need thereof, comprising administering to the animal an effective amount of an antigenic composition comprising a hybrid woodchuck hepadnavirus core antigen, wherein the hybrid core antigen is a fusion protein comprising a heterologous antigen and a woodchuck hepadnavirus core antigen with reduced antigenicity, and wherein said fusion protein assembles as a hybrid virus-like particle (VLP). Also provided by the present disclosure are methods for eliciting an immune response in an animal in need thereof, comprising administering to the animal an effective amount of an antigenic composition comprising a polynucleotide encoding a hybrid woodchuck hepadnavirus core antigen, wherein the hybrid core antigen is a fusion protein comprising a heterologous antigen and a woodchuck hepadnavirus core antigen with reduced antigenicity, and wherein said fusion protein assembles as a hybrid virus-like particle (VLP). Unless otherwise indicated, the antigenic composition is an immunogenic composition.

The immune response raised by the methods of the present disclosure generally includes an antibody response, preferably a neutralizing antibody response, preferably a protective antibody response. Methods for assessing antibody responses after administration of an antigenic composition (immunization or vaccination) are well known in the art. In some embodiments, the immune response comprises a T cell-mediated response (e.g., heterologous antigen-specific response such as a proliferative response, a cytokine response, etc.). In preferred embodiments, the immune response comprises both a B cell and a T cell response. Antigenic compositions can be administered in a number of suitable ways, such as intramuscular injection, subcutaneous injection, and intradermal administration. Additional modes of administration include but are not limited to intranasal administration, and oral administration.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Administration of more than one dose (typically two or three doses) is particularly useful in immunologically naive subjects or subjects of a hypo-responsive population (e.g., diabetics, subjects with chronic kidney disease, etc.). Multiple doses will typically be administered at least 1 week apart (e.g., about 2 weeks, about 3 weeks, about 4 weeks, about 6 weeks, about 8 weeks, about 10 weeks, about 12 weeks, about 16 weeks, and the like). Preferably multiple doses are administered from one, two, three, four or five months apart. Antigenic compositions of the present disclosure may be administered to patients at substantially the same time as (e.g., during the same medical consultation or visit to a healthcare professional) other vaccines.

In general, the amount of protein in each dose of the antigenic composition is selected as an amount effective to induce an immune response in the subject, without causing significant, adverse side effects in the subject. Preferably the immune response elicited is a neutralizing antibody, preferably a protective antibody response. Protective in this context does not necessarily mean the subject is completely protected against infection, rather it means that the subject is protected from developing symptoms of disease, especially severe disease associated with the pathogen corresponding to the heterologous antigen.

The amount of hybrid core antigen (e.g., VLP) can vary depending upon which antigenic composition is employed. Generally, it is expected that each human dose will comprise 1-1500 μg of protein (e.g., hybrid core antigen), such as from about 1 μg to about 1000 μg, for example, from about 1 μg to about 500 μg, or from about 1 μg to about 100 μg. In some embodiments, the amount of the protein is within any range having a lower limit of 10, 20, 30, 40, 50, 60, 70, 80, 90, 100, 110, 120, 130, 140, 150, 160, 170, 180, 190, 200, 210, 220, 230, 240 or 250 μg, and an independently selected upper limit of 1000, 950, 900, 850, 800, 750, 700, 650, 600, 550, 500, 450, 400, 350, 300 or 250 μg, provided that the lower limit is less than the upper limit. Generally a human dose will be in a volume of from 0.1 ml to 1 ml, preferably from 0.25 ml to 0.5 ml. The amount utilized in an immunogenic composition is selected based on the subject population. An optimal amount for a particular composition can be ascertained by standard studies involving observation of antibody titers and other responses (e.g., antigen-induced cytokine secretion) in subjects. Following an initial vaccination, subjects can receive a boost in about 4-12 weeks.

### Definitions

As used herein, the singular forms “a”, “an”, and “the” include plural references unless indicated otherwise. For example, “an” excipient includes one or more excipients. The term “plurality” refers to two or more.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Current Protocols in Molecular Biology (Ausubel et al., eds., 1987); PCR: The Polymerase Chain Reaction, (Mullis et al., eds., 1994); Culture of Animal Cells: A Manual of Basic Technique (Freshney, 1987); Harlow et al., Antibodies: A Laboratory Manual (Harlow et al., 1988); and Current Protocols in Immunology (Coligan et al., eds., 1991).

As used herein, the terms “virus-like particle” and “VLP” refer to a structure that resembles a virus. VLPs of the present disclosure lack a viral genome and are therefore noninfectious. Preferred VLPs of the present disclosure are woodchuck hepadnavirus core antigen (WHcAg) VLPs.

The terms “hybrid” and “chimeric” as used in reference to a hepadnavirus core antigen, refer to a fusion protein of the hepadnavirus core antigen and an unrelated antigen (e.g., bacterial polypeptide, and variants thereof). For instance, in some embodiments, the term “hybrid WHcAg” refers to a fusion protein comprising both a WHcAg component (full length, or partial) and a heterologous antigen or fragment thereof.

The term “heterologous” with respect to a nucleic acid, or a polypeptide, indicates that the component occurs where it is not normally found in nature and/or that it originates from a different source or species.

An “effective amount” or a “sufficient amount” of a substance is that amount necessary to effect beneficial or desired results, including clinical results, and, as such, an “effective amount” depends upon the context in which it is being applied. In the context of administering an immunogenic composition, an effective amount contains sufficient antigen (e.g., hybrid, WHcAg-hAg VLP) to elicit an immune response (preferably a measurable level of hAg pathogen-neutralizing antibodies). An effective amount can be administered in one or more doses.

The term “dose” as used herein in reference to an immunogenic composition refers to a measured portion of the immunogenic composition taken by (administered to or received by) a subject at any one time.

The term “about” as used herein in reference to a value, encompasses from 90% to 110% of that value (e.g., about 200 μg VLP refers to 180 μg to 220 μg VLP).

As used herein the term “immunization” refers to a process that increases an organisms' reaction to antigen and therefore improves its ability to resist or overcome infection.

The term “vaccination” as used herein refers to the introduction of vaccine into a body of an organism.

A “variant” when referring to a polynucleotide or a polypeptide (e.g., a viral polynucleotide or polypeptide) is a polynucleotide or a polypeptide that differs from a reference polynucleotide or polypeptide. Usually, the difference(s) between the variant and the reference constitute a proportionally small number of differences as compared to the reference (e.g., at least 90%, 91%, 92%, 93%, 94%, 95%, 96%, 97%, 98% or 99% identical). In some embodiments, the present disclosure provides hybrid WHcAg-hAg VLPs having at least one addition, insertion or substitution in one or both of the WHcAg or hAg portion of the VLP.

The term “wild type” when used in reference to a polynucleotide or a polypeptide refers to a polynucleotide or a polypeptide that has the characteristics of that polynucleotide or a polypeptide when isolated from a naturally-occurring source. A wild type polynucleotide or a polypeptide is that which is most frequently observed in a population and is thus arbitrarily designated as the “normal” form of the polynucleotide or a polypeptide.

Amino acids may be grouped according to common side-chain properties: hydrophobic (Met, Ala, Val, Leu, Ile); neutral hydrophilic (Cys, Ser, Thr, Asn, Gin); acidic (Asp, Glu); basic (His, Lys, Arg); aromatic (Trp, Tyr, Phe); and orientative (Gly, Pro). Another grouping of amino acids according to side-chain properties is as follows: aliphatic (glycine, alanine, valine, leucine, and isoleucine); aliphatic-hydroxyl (serine and threonine); amide (asparagine and glutamine); aromatic (phenylalanine, tyrosine, and tryptophan); acidic (glutamic acid and aspartic acid); basic (lysine, arginine, and histidine); sulfur (cysteine and methionine); and cyclic (proline). In some embodiments, the amino acid substitution is a conservative substitution involving an exchange of a member of one class for another member of the same class. In other embodiments, the amino acid substitution is a non-conservative substitution involving an exchange of a member of one class for a member of a different class.

The percent identity between the two sequences is a function of the number of identical positions shared by the sequences, taking into account the number of gaps, and the length of each gap, which need to be introduced for optimal alignment of the two sequences. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=1 and Gap extension penalty=1.

A “recombinant” nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques. A “recombinant” protein is one that is encoded by a heterologous (e.g., recombinant) nucleic acid, which has been introduced into a host cell, such as a bacterial or eukaryotic cell. The nucleic acid can be introduced, on an expression vector having signals capable of expressing the protein encoded by the introduced nucleic acid or the nucleic acid can be integrated into the host cell chromosome.

An “antigen” is a compound, composition, or substance that can stimulate the production of antibodies and/or a T cell response in a subject, including compositions that are injected, absorbed or otherwise introduced into a subject. The term “antigen” includes all related antigenic epitopes. The term “epitope” or “antigenic determinant” refers to a site on an antigen to which B and/or T cells respond. The “dominant antigenic epitopes” or “dominant epitope” are those epitopes to which a functionally significant host immune response, e.g., an antibody response or a T-cell response, is made. Thus, with respect to a protective immune response against a pathogen, the dominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

“Adjuvant” refers to a substance which, when added to a composition comprising an antigen, nonspecifically enhances or potentiates an immune response to the antigen in the recipient upon exposure. Common adjuvants include suspensions of minerals (alum, aluminum hydroxide, aluminum phosphate) onto which an antigen is adsorbed; emulsions, including water-in-oil, and oil-in-water (and variants thereof, including double emulsions and reversible emulsions), liposaccharides, lipopolysaccharides, immunostimulatory nucleic acids (such as CpG oligonucleotides), liposomes, Toll-like Receptor agonists (particularly, TLR2, TLR4, TLR7/8 and TLR9 agonists), and various combinations of such components.

An “antibody” or “immunoglobulin” is a plasma protein, made up of four polypeptides that binds specifically to an antigen. An antibody molecule is made up of two heavy chain polypeptides and two light chain polypeptides (or multiples thereof) held together by disulfide bonds. In humans, antibodies are defined into five isotypes or classes: IgG, IgM, IgA, IgD, and IgE. IgG antibodies can be further divided into four subclasses (IgG1, IgG2, IgG3 and IgG4). A “neutralizing” antibody is an antibody that is capable of inhibiting the infectivity of a virus. Accordingly, a neutralizing antibodies specific for a virus are capable of inhibiting or reducing infectivity of the virus.

An “immunogenic composition” is a composition of matter suitable for administration to a human or animal subject (e.g., in an experimental or clinical setting) that is capable of eliciting a specific immune response, e.g., against a pathogen, such as a malaria parasite. As such, an immunogenic composition includes one or more antigens (for example, polypeptide antigens) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or reduced or ameliorated) by inhibiting replication of the pathogen (e.g., virus) following exposure of the subject to the pathogen. In the context of this disclosure, the term immunogenic composition will be understood to encompass compositions that are intended for administration to a subject or population of subjects for the purpose of eliciting a protective or palliative immune response against a virus (that is, vaccine compositions or vaccines).

An “immune response” is a response of a cell of the immune system, such as a B cell, T cell, or monocyte, to a stimulus, such as a pathogen or antigen (e.g., formulated as an immunogenic composition or vaccine). An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo. Exposure of a subject to an immunogenic stimulus, such as a pathogen or antigen (e.g., formulated as an immunogenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an immunogenic composition increases the magnitude of an antigen (or pathogen) specific response, (e.g., by increasing antibody titer and/or affinity, by increasing the frequency of antigen specific B or T cells, by inducing maturation effector function, or any combination thereof).

The term “reduces” is a relative term, such that an agent reduces a response or condition if the response or condition is quantitatively diminished following administration of the agent, or if it is diminished following administration of the agent, as compared to a reference agent. Similarly, the term “protects” does not necessarily mean that an agent completely eliminates the risk of an infection or disease caused by infection, so long as at least one characteristic of the response or condition is substantially or significantly reduced or eliminated. Thus, an immunogenic composition that protects against or reduces an infection or a disease, or symptom thereof, can, but does not necessarily prevent or eliminate infection or disease in all subjects, so long as the incidence or severity of infection or incidence or severity of disease is measurably reduced, for example, by at least about 50%, or by at least about 60%, or by at least about 70%, or by at least about 80%, or by at least about 90% of the infection or response in the absence of the agent, or in comparison to a reference agent.

A “subject” refers to a mammalian subject. In the context of this disclosure, the subject can be an experimental subject, such as a non-human mammal (e.g., mouse, rat, rabbit, non-human primate, etc.). Alternatively, the subject can be a human subject.

The terms “derived from” or “of” when used in reference to a nucleic acid or protein indicates that its sequence is identical or substantially identical to that of an organism of interest.

The terms “decrease,” “reduce” and “reduction” as used in reference to biological function (e.g., enzymatic activity, production of compound, expression of a protein, etc.) refer to a measurable lessening in the function by preferably at least 10%, more preferably at least 50%, still more preferably at least 75%, and most preferably at least 90%. Depending upon the function, the reduction may be from 10% to 100%. The term “substantial reduction” and the like refers to a reduction of at least 50%, 75%, 90%, 95% or 100%.

The terms “increase,” “elevate” and “elevation” as used in reference to biological function (e.g., enzymatic activity, production of compound, expression of a protein, etc.) refer to a measurable augmentation in the function by preferably at least 10%, more preferably at least 50%, still more preferably at least 75%, and most preferably at least 90%. Depending upon the function, the elevation may be from 10% to 100%; or at least 10-fold, 100-fold, or 1000-fold up to 100-fold, 1000-fold or 10,000-fold or more. The term “substantial elevation” and the like refers to an elevation of at least 50%, 75%, 90%, 95% or 100%.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell (e.g., bacteria) that produced the protein. As such an isolated protein is free of extraneous compounds (e.g., culture medium, bacterial components, etc.).

### ENUMERATED EMBODIMENTS

1. An antigenic composition comprising a hybrid hepadnavirus core antigen, wherein
the hybrid core antigen is a fusion protein comprising a first portion of a human hepatitis B virus surface antigen (HBsAg) and a woodchuck hepadnavirus core antigen (WHcAg),
the first portion of the HBsAg consists of from 8 to 50 amino acids of the PreS1 domain of the human hepatitis B virus (HBV) large surface antigen,
the amino acid sequence of the WHcAg is at least 95% identical to SEQ ID NO:1,
the amino acid sequence of the PreS1 domain is at least 95% identical to SEQ ID NO:7 or SEQ ID NO:41,
the first portion of the HBsAg is inserted at a first position,
the first position is N-terminus or an internal position of the WHcAg selected from the group consisting of 61, 71, 72, 73, 74, 75, 76, 77, 78, 81, 82, 83, 84, 85 and 92 as numbered according to SEQ ID NO:1, and
the fusion protein is capable of assembling as a hybrid PreS1-WHcAg virus-like particle (VLP).

2. The antigenic composition of embodiment 1, wherein the first position is an internal position of the core antigen selected from the group consisting of 61, 71, 72, 73, 74, 75, 76, 77, 78, 81, 82, 83, 84, 85 and 92 as numbered according to SEQ ID NO:1, optionally wherein the first position is position 78.

3. The antigenic composition of embodiment 1, wherein the hybrid core antigen further comprises a second portion of the HBsAg consisting of from 8 to 50 amino acids in length of the PreS1 domain of the large surface antigen, the second portion is inserted at a second position, and the second position is N-terminus or an internal position of the WHcAg selected from the group consisting of 61, 71, 72, 73, 74, 75, 76, 77, 78, 81, 82, 83, 84, 85 and 92 as numbered according to SEQ ID NO:1.

4. The antigenic composition of embodiment 3, wherein the amino acid sequence of the second portion of the HBsAg is different than the amino acid sequence of the first portion of the HBsAg.

5. The antigenic composition of embodiment 3 or embodiment 4, wherein the second position is the N-terminus.

6. The antigenic composition of embodiment 3 or embodiment 4, wherein the first position is 78 and the second position is the N-terminus.

7. The antigenic composition of embodiment 3 or embodiment 4, wherein the first position is adjacent to the second position, and the first portion and the second portion together are no more than 50 amino acids in length.

8. The antigenic composition of embodiment 7, wherein the first portion is inserted at position 78 and the second portion is inserted at the C-terminus of the first portion or at the C-terminus of intervening sequence separating the first portion from the second portion, optionally wherein the intervening sequence comprises GGGG (SEQ ID NO:31) or EEEE (SEQ ID NO:30).

9. The antigenic composition of any one of embodiments 1-8, wherein the first portion is inserted at an internal site as a linker/insert combination according to the formula GIL(E)y-Xn-(E)zL (SEQ ID NO:29, in which both y and z are in integers independently selected from the group consisting of 0, 1, and 2, and wherein Xn is the first portion.

10. The antigenic composition of any one of embodiments 1-9, wherein the WHcAg has a serine at position 61.

11. The antigenic composition of any one of embodiments 1-9, wherein the WHcAg as a cysteine at position 61.

12. The antigenic composition of any one of embodiments 1-11, wherein the amino acid sequence of:
i) one or both of the first portion and the second portion each comprise one of the group consisting of SEQ ID NOs:13-24; or
ii) one or both of the first portion and the second portion are each at least 95% identical one of the group consisting of SEQ ID NOs:13-24; or
iii) the first portion is selected from the group consisting of SEQ ID NO:15, SEQ ID NO:16, SEQ ID NO:17, and SEQ ID NO:21; or
iv) the second portion is selected from the group consisting of SEQ ID NO:15 and SEQ ID NO:17.

13. The antigenic composition of embodiment 1, wherein the amino acid sequence of the hybrid PreS1-WHcAg VLP is at least 95% identical to one of the group consisting of SEQ ID NO:32, SEQ ID NO:33, SEQ ID NO:34 and SEQ ID NO:35.

14. The antigenic composition of any one of embodiments 1-13, wherein the hybrid PreS1-WHcAg VLP elicits an antibody response against one or more of HBV virions, HBsAg particles, a PreS1 protein consisting of the amino acid sequence of SEQ ID NO:7, and a PreS1+S2 protein consisting of the amino acid sequence of SEQ ID NO:9.

15. The antigenic composition of any one of embodiments 1-14, wherein the hybrid PreS1-WHcAg VLP elicits a measurable neutralizing antibody response against HBV.

16. An antigenic composition comprising a hybrid hepadnavirus core antigen, wherein
the hybrid core antigen is a fusion protein comprising a human hepatitis B virus core antigen (HBcAg) and a woodchuck hepadnavirus core antigen (WHcAg), and
the fusion protein is capable of assembling as a hybrid HBcAg-WHcAg virus-like particle (VLP).

17. The antigenic composition of embodiment 16, wherein the amino acid sequence of the HBcAg is at least 95% identical to SEQ ID NO:4, and the amino acid sequence of the WHcAg is at least 95% identical to SEQ ID NO:1.

18. The antigenic composition of embodiment 16 or embodiment 17, wherein a dimer linker of from 5-15 amino acids in length is inserted between the amino acid sequence of the HBcAg and the amino acid sequence of the WHcAg, optionally wherein the dimer linker comprises the amino acid sequence of SEQ ID NO:38 or SEQ ID NO:39.

19. The antigenic composition of embodiment 16, wherein the amino acid sequence of the hybrid HBcAg-WHcAg virus-like particle (VLP) is at least 95% identical to SEQ ID NO:36 or SEQ ID NO:37.

20. A vaccine comprising the antigenic composition of any one of embodiments 1-19, and an adjuvant.

21. A polynucleotide encoding the hybrid hepadnavirus core antigen of any one of embodiments 1-15.

22. An expression construct comprising the polynucleotide of embodiment 21 in operable combination with a promoter, optionally wherein the promoter drives expression of the hybrid hepadnavirus core antigen in bacterial cells.

23. An expression vector comprising the expression construct of embodiment 22.

24. A polynucleotide encoding the hybrid hepadnavirus core antigen of any one of embodiments 16-19.

25. An expression construct comprising the polynucleotide of embodiment 24 in operable combination with a promoter, optionally wherein the promoter drives expression of the hybrid hepadnavirus core antigen in mammalian cells.

26. An expression vector comprising the expression construct of embodiment 25.

27. A host cell comprising the expression vector of embodiment 23 or embodiment 26, optionally wherein the nucleic acid sequence of the expression construct is optimized for expression in bacterial cells or mammalian cells.

28. A method for eliciting or enhancing an HBsAg-reactive antibody response, the method comprising:
administering to a mammalian subject an effective amount of a vaccine comprising an adjuvant and the antigenic composition of any one of embodiments 1-15.

29. The method of embodiment 28, wherein the HBsAg-reactive antibody response comprises antibodies reactive with one or more of HBV virions, HBsAg particles, a PreS1 protein consisting of the amino acid sequence of SEQ ID NO:7, and a PreS1+S2 protein consisting of the amino acid sequence of SEQ ID NO:9.

30. A method for eliciting or enhancing a HBcAg-reactive T lymphocyte response, the method comprising:
administering to a mammal subject an effective amount of the expression vector of embodiment 25.

31. The method of embodiment 30, wherein the HBcAg-reactive T lymphocyte response comprises:
i) interferon-gamma secretion inducible by presentation of HBcAg-derived peptides by antigen presenting cells of the mammalian subject; and
ii) HBcAg-specific cytotoxic T lymphocytes.

32. A method for eliciting or enhancing an HBsAg-reactive antibody response and a HBcAg-reactive T lymphocyte response, the method comprising administering to a mammalian subject:
an effective amount of a vaccine comprising an adjuvant and the antigenic composition of any one of embodiments 1-15; and
an effective amount of the expression vector of embodiment 25.

33. The method of embodiment 32, wherein the vaccine and the expression vector are administered concurrently or an separate occasions.

34. The method of embodiment 33, wherein the vaccine and the expression vector are each administered on 1, 2 or 3 occasions.

35. The method of embodiment 34, wherein the vaccine and the expression vector are each administered at 1, 2, 3, 4, 5 or 6 month intervals, optionally at 1 or 2 month intervals.

36. The method of embodiment 33, wherein the vaccine is administered intramuscularly, intradermally or subcutaneously, and the expression vector is administered intramuscularly.

37. The method of embodiment 28 or embodiment 29, or any one of embodiments 32-36, wherein the antigenic composition comprising a plurality hybrid PreS1-WHcAg VLPs, wherein the plurality comprises 2, 3, or 4 different hybrid PreS1-WHcAg VLPs.

38. The method of embodiment 37, wherein the amino acid sequences of the 2, 3, or 4 different hybrid PreS1-WHcAg VLPs are each at least 95% identical to one of the group consisting of SEQ ID NO:32, SEQ ID NO:33, SEQ ID NO:34 and SEQ ID NO:35.

39. The method of any one of embodiments 28-38, wherein the mammalian subject is chronically-infected with HBV.

40. The method of embodiment 39, wherein the mammalian subject is HBeAg-positive.

41. The method of any one of embodiments 28-38, wherein the mammalian subject is a low or non-responder to a preventative vaccine comprising HBsAg and an aluminum salt.

42. The method of any one of embodiments 28-38, wherein the mammalian subject is a pregnant HBV-positive carrier.

### EXAMPLES

In the experimental disclosure below, the following abbreviations apply: Ab (antibody); BSA (bovine serum albumin); cccDNA (covalently-closed circular DNA); CTL (cytotoxic T lymphocyte); ELISA (enzyme-linked immunosorbent assay); (hAg) heterologous antigen; HBcAg (hepatitis B virus core antigen); HBsAg (hepatitis B virus surface antigen); HBV (hepatitis B virus); IFA (incomplete Freund's adjuvant); IM (intramuscular); IP (intraperitoneal); MAb (monoclonal antibody); nAb (neutralizing antibody); OD (optical density); PBS (phosphate buffered saline); SC (subcutaneous); (Th) helper; VLP (virus-like particle); and WHcAg (woodchuck hepadnavirus core antigen).

Although, the present disclosure has been described in some detail by way of illustration and example for purposes of clarity and understanding, it will be apparent to those skilled in the art that certain changes and modifications may be practiced. Therefore, the following examples should not be construed as limiting the scope of the present disclosure, which is delineated by the appended claims.

### Example 1: Therapeutic Hepatitis B Vaccine

This example describes the production and analysis of a therapeutic hepatitis B vaccination regimen involving a virus-like particle (VLP) prime and a DNA boost to elicit an HBV-neutralizing antibody (nAb) response and HBcAg-specific CD4+ helper T (Th) cell and CD8+ cytotoxic T lymphocyte (CTL) responses.

### Materials & Methods

Mice: Inbred C57BL/6 (B6) (H-2^{b}), C57BL/10 (B10) (H-2^{b}), B6/BALBc (H-2^{bxd}) mice and B10, TLR7-KO mice were obtained from The Jackson Laboratory. B10-Tg mice with intrahepatic expression of the HBcAg protein (HBc-Tg, 0.2 to 2 μg/mg liver protein) or the HBeAg protein (HBe-Tg, 4 to 10 μg/ml serum were obtained from Dr. J. Ou of the University of Southern California (Chen 2004a; Guidotti 1996a). The HBV-Tg mice (1.3.32) were obtained from F. Chisari (Guidotti 1995), and PreS1-Tg mice (lineage 107-5) were provided by F. Chisari and L. Guidotti of (Nakamoto 1998), both of The Scripps Research Institute.

Recombinant WHcAg hybrid VLP Construction: The WHcAg and hybrid WHcAg VLPs were expressed from the pUC-WHcAg vector expressing the full-length WHcAg protein codon optimized for expression in E. coli. The sequence for WHcAg (accession NC_004177) was cloned into the pUC19 vector. For inserting heterologous B cell epitopes, EcoRI-XhoI restriction sites were engineered into the open reading frame between amino acids 78 and 79 of the core protein gene. The engineered restriction sites add a Gly-Ile-Leu linker on the N-terminal side and a Leu linker on the C-terminal side of the inserted epitopes. For VLP-1.6 the heterologous B cell epitope was directly fused between amino acids 78 and 79 by the polymerase chain reaction using overlapping primers. For fusing the heterologous T950-969 T cell epitope, the Aval restriction site was used in the HyW VLP previously described (Billaud, 2005). Epitopes were cloned into the VLP gene using synthetic oligonucleotides comprising the desired epitope coding sequence and the appropriate engineered restriction sites or overlapping primers. VRI010c (aka FLw-HBV-PreS2-74x3-UTC) was constructed by inserting three copies of the PreS2 epitope into the VLP open reading frame at amino acid position 74 using an existing SacI site. Additionally, the sequence DIEYLNKIQNSLSTEWSPCSVT (SEQ ID NO:40) was fused to the C-terminus of the WHcAg using an EcoRV site engineered after the terminal (C188). VLPs were produced, immunized, and virus neutralization tested as described herein. All WHcAg constructs were transformed into Alpha-Select competent E. coli (Bioline USA, Inc.) and confirmed by DNA sequencing.

The VLP constructs delivered as DNA were codon optimized for mammals and cloned into pVAX1, grown in Alpha-Select cells and plasmid purified by the ZymoPure II MaxiPrep kit (D4202, Zymo Research) according to the manufacturer's instructions. DNA was formulated in PBS and concentration was determined spectrophotometrically.

Purified Proteins, Synthetic Peptides and Mabs: The VLP particles were expressed in Alpha-Select E. coli cells grown in Terrific Broth (Fisher BP2468). Cells were lysed by passage through an EmulsiFlex-C3 (Avestin, Ottawa, ON, Canada) and the lysate heated to 65° C. for approximately 10 min, then clarified by centrifugation. The WHcAg particles were selectively precipitated by the addition of solid ammonium sulfate up to approximately 45% saturation (277 g/L) and the precipitates were collected by centrifugation. Precipitated VLPs were re-dissolved in minimum buffer (10 mM Tris, pH 8), dialyzed against the same buffer and applied to a Sepharose CL4B column (5×100 cm) or ultra-filtered by tangential flow using a WaterSep Discover 12, 750 K molecular weight cutoff. Finally, VLPs were formulated in 20 mM Tris, pH 8, 100 mM NaCl, 0.5 mM EDTA. Endotoxin was removed from the core preparations by phase separation with Triton X-114 (Billaud 2005; Aida 1990). The purified VLPs were 0.2 μm sterile-filtered, characterized and aliquoted. Characterization typically included custom ELISA, native agarose gel electrophoresis PAGE, heat stability, circular dichroism and dynamic light scattering as previously described (Billaud 2005).

To produce rPreS1+2 and myr-PreS1+2, the gene encoding ayw preS1S2 (aa1-163) fused to a six-histidine tag was cloned alone or together with the yeast N-methyltransferase 1 gene into the pET Duet vector and transformed into HMS174(DE3) E. coli. Bacteria were grown on LB medium supplemented with 2 g/l glucose, at 30° C., until the A₆₀₀ was between 0.6 and 0.8. In the case of the dual expression cells, the medium was then supplemented with 10 ml of 5 mM mystistic acid in 0.6 mM BSA in water. At this time 100 mg of IPTG was added per liter of culture, and the bacteria allowed to continue to grow for 3-4 hours. Bacteria were collected by centrifugation and stored frozen until processed. Bacterial pellets were suspended in 6M urea and disrupted by a single passage through an Avestin Emulsiflex C3 operating at a pressure of 25000 psi. The use of urea was to prevent the rapid proteolysis of the soluble protein upon disruption of the bacteria. The lysate was clarified by centrifugation and applied to a nickel column (BioRad), extensively washed with 6 M urea until the absorbance at 280 nm had returned to baseline values then washed with water to remove the urea. The protein was eluted using 50 mM citric acid and dialyzed against 10 mM acetate buffer pH 5.0.

Synthetic peptides were synthesized by and purchased from Eton Biosciences (San Diego, Calif.) and Abclonal (Woburn, Mass.).

PreS1-specific Mab 18/7 was provided by W. Gerlich of Justus Liebig University Giessen. PreS1-specific Mabs AP-2 and KD-127 were purchased from Santa Cruz Biotechnology (Dallas, Tex.). PreS1-specific Mab Ab001 was purchased from Beacle, Inc. (Okayama, Japan).

Human-liver chimeric mice: Homozygous NRG-fumarylacetoacetate hydrolase (fah/fah) mutant mice (NRG/F) were maintained with 8 μg/ml 2-(2-nitro-4-fluromethylbenzoyl)-1,3 cyclohexanedine (NTBC) (Li 2014). Anesthetized mice were injected in the spleen with 1×10⁶ human primary hepatocytes (Triangle Research Laboratories, NC). After transplantation the NRG/F-hu mice were subjected to three rounds of NTBC drug recycling to eliminate mouse hepatocytes and to provide space for human hepatocyte growth. Thereafter, mice were infected with 1×10⁶ GE copies of HBV/genotype C (isolated from HBV-infected human-liver chimeric mouse serum) injected retro-orbitally. Human albumin in mouse sera was measured with a modified ELISA method (Bethyl Labs Human Albumin ELISA Quantitation Set)(Li 2017).

Passive transfer of Antisera: Anti-PreS1-WHc VLP sera (0.2 ml) or control anti-WHc sera (0.2 ml) were injected IV into human-liver chimeric mice prior to infection with 1×10⁶ GE copies of HBV per mouse in the control and acute groups. For the chronic group, 0.2 ml of anti-PreS1-WHc VLP sera were injected (IV) 2 and 5 weeks after HBV infection. To measure HBV DNA, sera were collected from the tail vein and HBV DNA was extracted with QIAamp MinElute Virus Spin Kit according to the manufacturer's instructions. Primer 1 (HBV2270F: 5′-GAGTGTGGATTCGCACTCC-3′ set forth as SEQ ID NO:27) and Primer 2 (HBV2392R: 5′-GAGGCGAGGGAGTTCTTCT-3′ set forth as SEQ ID NO:28) were used in the Q-PCR reaction to measure HBV DNA. A human serum with known viral titer was used as an HBV DNA standard (Li 2017).

Virus neutralization assay: Neutralization was assessed as previously described (Blanchet 2006). Briefly, HDV (HBV genotype D, L/M/S-HBsAg subtype ayw) particles were derived from the culture medium from transfected cells and suspended at 1×10⁹ particles per ml. 100 μl of HDV-HBV particles were mixed with 100 μl of sample (sera or purified Mab neat, 1/10 and 1/100 dilutions in PBS) and incubated at 37° C. for 1 hour such that final dilutions of sera were 1:40, 1:400 and 1:4000 and Mab was 5, 0.5 and 0.05 μg. The mix was adjusted to 5% PEG and inoculated on HepaRG (10⁶ cells for a multiplicity of infection of 100 viral particles per hepatocyte). After 16 hours, the inoculum was removed and replaced with fresh medium. Cells were harvested at day 7 post inoculation for detection of intracellular genomic HDV RNA as a marker of infection by Northern Blot and quantified by densitometry.

Immunizations and serology: Groups of mice were immunized intraperitoneally (IP) with the PreS1-WHc VLPs (usually 10-20 μg) emulsified in incomplete Freund's adjuvant (IFA) for both antibody production and T cell experiments. DNA immunization was performed intramuscularly in the tibialis cranialis muscle with 50 μg pVAX-VLP-1.6 plasmid in a volume of 50 μl. Immediately after immunization the muscle was electroporated using Clinporator 2 device (IGEA, Carpi, Italy) with a pulse pattern of one 1 ms 600 V/cm pulse followed by a 400 mg 60 V/cm pulse. A booster dose was given one month later and mice were sacrificed two weeks thereafter. For antibody experiments, mice were bled retro-orbitally and sera pooled from each group. Periodically individual mouse sera were tested to confirm the fidelity of the pooled sera results. Anti-WHc and anti-insert IgG antibodies were measured in murine sera by an indirect solid-phase ELISA by using the homologous WHcAg (50 ng/well), HBV virions, rPreS1+2, or synthetic peptides (0.5 μg/well), representing the inserted PreS1 sequences, as solid-phase ligands as described previously (Milich 1986a). Serial dilutions of both test sera and pre-immunization sera were made and the data are expressed as antibody titers representing the reciprocals of the highest dilutions of sera required to yield an optical density at 492 nm (OD 492) three times an equal dilution of pre-immunization sera.

In vitro T cell cytokine assays: Spleen cells from groups of 3 mice each of the various lineages were harvested and pooled 4-6 weeks after immunization with the PreS1-WHc VLPs. Spleen cells (5×10⁵) were cultured with varying concentrations of WHcAg, HBcAg or synthetic peptides derived from the WHcAg, HBcAg or PreS region. For cytokine assays, culture supernatants were harvested at 48 h for IL-2 determination and at 96 h for interferon-gamma (IFNγ) determination by ELISA. IFNγ production was measured by a two-site ELISA using mAb 170 and a polyclonal goat anti-mouse IFNγ (Genzyme Corp., Boston, Mass.).

### Results

Construction of PreS1-WHc VLPs. The HBV genome encodes three envelope proteins termed small (S), middle (M) and large (L) proteins, which share the C-terminal HBsAg domain. The M and L proteins carry additional N-terminal extensions of 55aa (PreS2 region) and 108 or 119aa depending on genotype (PreS1 region). The 1-108aa (genotype D) PreS1 sequence numeration is used throughout. The stoichiometric ratio of UM/S proteins in HBV virions is approximately 1:1:4, whereas the most abundant secreted small noninfectious subviral particles contain almost exclusively the small HBsAg protein, lesser amounts of PreS2 and only trace amounts of the PreS1 region (Heermann 1984). Therefore, for a therapeutic HBV vaccine designed to elicit virion-specific antibodies, inclusion of PreS B cell epitopes is imperative. Antibodies produced to the HBsAg domain can be “absorbed-out” by subviral HBsAg particles that circulate at levels as high as 1.0 mg/ml in chronic HBV sera. A second imperative is that the PreS B cell epitopes chosen must elicit HBV-specific nAbs. A number of PreS1-specific B cell epitopes have been identified in mice by immunization with HBsAg/L particles. PreS1-derived synthetic peptides and by serological analysis of human HBV-infected blood samples (i.e., 1-21, 21-47 and 83-106) (Milich 1986a; Alberti 1990). Further, in vitro neutralization studies and in vivo immunization studies with PreS1 epitope-specific synthetic peptides demonstrated that PreS1-specific antibodies could protect chimpanzees from experimental HBV challenge in the absence of anti-HBs region antibody (Neurath 1989; Neurath 1987; Thornton 1989). Subsequently, several groups delineated the PreS1 residues (aa 9-18) and (aa28-48) involved in HBV-hepatocyte receptor recognition (Barrera 2005; Glebe 2005; Neurath 1986).

Four PreS1 B cell epitopes (1.1, 1.2, 1.3 and 1.4; see Table 1) were initially chosen for insertion onto the exposed loop region of the WHcAg platform. Four PreS1-WHc VLPs were selected from a larger library based on assembly, yield, and antigenicity determined by binding to a series of PreS1-specific monoclonal antibodies (Mabs). The inserted PreS1 sequences were modified in a second set of VLPs designated 1.1+, 1.3+, 1.4+ and 1.5 in order to broaden recognition by the panel of PreS1-specific Mabs. As shown in Table 1, purified HBV virions, recombinant (r) PreS1/PreS2 protein and myristoylated rPreS1/PreS2 protein were recognized by all four PreS1-specific Mabs and an anti-PreS1 peptide (aa83-106)-reactive polyclonal antisera. Mab Ab001, which binds aa1-15 was not blocked by myristolation as previously suggested, indicating heterogeneity in the Ab response to the PreS1 amino terminus (Bremer 2011). The PreS1-specific Mab binding profiles for the selected PreS1-WHc VLPs and a series of synthetic peptides demonstrated that the inserted PreS1 sequences were accessible on the surface of the VLPs and appropriately antigenic. Based on antigenicity and immunogenicity data, the inserted PreS1 sequences from VLP-1.1+ and VLP-1.4+ were consolidated into the loop region of a single VLP (1.6). The PreS1 sequences from VLP1.3+ (inserted into the WHcAg loop) and VLP1.2 (fused to the N-terminus of the WHcAg) were also consolidated onto a single VLP (1.9). The combination of VLPs 1.6+1.9 was efficiently bound by all four PreS1-specific Mabs and the anti-aa83-106 antisera in a solid phase ELISA format (Table 1).

TABLE 1
Antigenicity of PreS1-WHc VLPs

| | PreS1-Specific Mabs (Min binding cone. [ng/ml]) | | | | |
|---|---|---|---|---|---|
| | Ab00l | 18/7 | AP-2 | KR-127 | α-p(83-106) |
| | aa(1-15) | aa(20-23) | aa(23-34) | aa(26-34) | aa(83-106) |
| Antigens | | | | | |
| HBV (virions) | 0.5 | 2.0 | 25.0 | 75.0 | + |
| rPreS1+2 | 0.3 | 0.3 | 1.6 | 1.6 | + |
| myr-rPreS1+2 | 0.3 | 0.3 | 1.6 | 1.6 | + |
| PreS1-VLPs | | | | | |
| VLP-1.1 (21-43) | 0 | 0 | 1.6 | 1.6 | 0 |
| VLP-1.1+ (19-43) | 0 | 0.3 | 40 | 1,000 | 0 |
| VLP-1.2 (83-106) | 0 | 0 | 0 | 0 | + |
| VLP-1.3 (1-21) | 0.3 | 0 | 0 | 0 | 0 |
| VLP-1.3+ (1-25) | 0.3 | 1.6 | 0 | 0 | 0 |
| VLP-1.4 (15-31) | 0 | 0.3 | 0 | 0 | 0 |
| VLP-1.4+ (15-34) | 0 | 8.0 | 8.0 | 8.0 | 0 |
| VLP-1.5 (1-34) | 0.3 | 0.3 | 0 | 0 | 0 |
| VLP-1.6 (15-40) | 0 | 0.3 | 1.6 | 1.6 | 0 |
| VLP-1.9 (83-106-NH2/ | 0.3 | 1.6 | 0 | 0 | + |
| 1-25-loop) | | | | | |
| VLPs 1.6+1.9 | 0.3 | 0.3 | 1.6 | 1.6 | + |
| PreS1-Peptides | | | | | |
| p(1-15) | 20 | 0 | 0 | 0 | 0 |
| p(5-16) | 1.6 | 0 | 0 | 0 | 0 |
| p(11-21) | 0 | 0 | 0 | 0 | 0 |
| p(18-25) | 0 | 8.0 | 0 | 0 | 0 |
| p(1-36) | 0.3 | 1.6 | 8.0 | 1.6 | 0 |
| p(21-42) | 0 | 0 | 8.0 | 1.6 | 0 |
| p(83-106) | 0 | 0 | 0 | 0 | + |

Immunogenicity of PreS1-WHc VLPs. Each PreS11-WHc VLP was analyzed for immunogenicity and anti-PreS11 Ab fine specificity by injecting groups of B10 mice each with 20 μg and boosting with 10 μg of VLP formulated in incomplete Freund's adjuvant (IFA) (Table 2). All VLPs elicited anti-PreS1 IgG endpoint titers of at least 1:625,000 after a boost measured on rPreS1/2 protein and the titers ranged between 1:125,000) to 1:6×10⁶ (i.e., VLP1.6) as measured on purified HBV virions, demonstrating the relevance of the anti-PreS11 Ab response to the virus. The fine specificity of the anti-PreS1 Abs correlated well with the PreS11 sequences inserted on the VLPs. For example, VLP-1.2 elicited Abs specific for only aa83-106, the inserted sequence; and VLP-1.3 elicited Abs specific for the aa 1-15 insert. Further fine specificity mapping of the anti-VLP antisera on a large panel of PreS1 peptides (Table 3) revealed that the PreS1 B cell epitopes segregated into an N-terminal domain (aa1-21: containing three overlapping epitopes, aa1-15, aa5-16 and aa11-21); a central domain (aa20-47: containing three overlapping epitopes, aa20-23, aa21-32, and aa3-42); and a C-terminal domain (aa83-106: containing two epitopes, aa83-94 and aa95-106) revealing at least 8 PreS1 epitopes identified by antisera raised against this panel of 10 PreS1-WHc VLPs. VLPs 1.6 and 1.9 elicited a complimentary set of PreS1-specific Abs and combining these two VLPs for immunization yielded the full spectrum of anti-PreS1 Ab specificities (Table 3).

TABLE 2
Immunogenicity and Fine Specificity of Anti-PreS-WHc VLP Sera

| | | | Antibody titer (1/dilution) vs PreS1 Antigens | | | | |
|---|---|---|---|---|---|---|---|
| anti-VLP Sera | HBV¹ | rPreS1+2 | p(1-15) | p(18-25) | p(1-36) | p(21-42) | p(83-106) |
| VLP-1.1 | 125K² | 625K | 0 | 0 | 625K | 3 × 10⁶ | 0 |
| VLP-1.1+ | 125K | 3 × 10⁶ | 0 | 125K | 6 × 10⁶ | 125K | 0 |
| VLP-1.2 | 625K | 625K | 0 | 0 | 0 | 0 | 625K |
| VLP-1.3 | 125K | 625K | 3 × 10⁶ | 0 | 3 × 10⁶ | 0 | 0 |
| VLP-1.3+ | 625K | 3 × 10⁶ | 625K | 5K | 3 × 10⁶ | 0 | 0 |
| VLP-1.5 | 125K | 625K | 50K | 10K | 2.1 × 10⁶ | 50K | 0 |
| VLP-1.4 | 625 K | 625K | 0 | 100K | 1.2 × 10⁶ | 5K | 0 |
| VLP-1.4+ | 3 × 10⁶ | 3 × 10⁶ | 0 | 50K | 6.4 × 10⁶ | 625 K | 0 |
| Hybrid VLPs | | | | | | | |
| VLP-1.6 | 6 × 10⁶ | 6 × 10⁶ | 0 | 625K | 6.4 × 10⁶ | 3 × 10⁶ | 0 |
| VLP-1.9 | 125K | 1.2 × 10⁶ | 125K | 5K | 625K | 0 | 125K |

TABLE 3
Immunogenicity and Fine Specificity of the Combination of VLP-1.6 and VLP-1.9

| | Antibody Titer (1/dilution) vs. PreS1 Antigens | | | | | |
|---|---|---|---|---|---|---|
| Immunogen | WHc | rPreS1 + 2 | p(1-15) | p(5-16) | p(11-21) | p(18-25) |
| VLP-1.6 + VLP-158 | 250K | 2 × 10⁶ | 57K | 34K | 32K | 0.5K |
| Immunogen | p(1-36) | p(22-32) | p(30-42) | p(21-42) | p(83-106) | HBV |
| VLP-1.6 + VLP-158 | 625 K | 0.8K | 6K | 250K | 23 K | 37K |

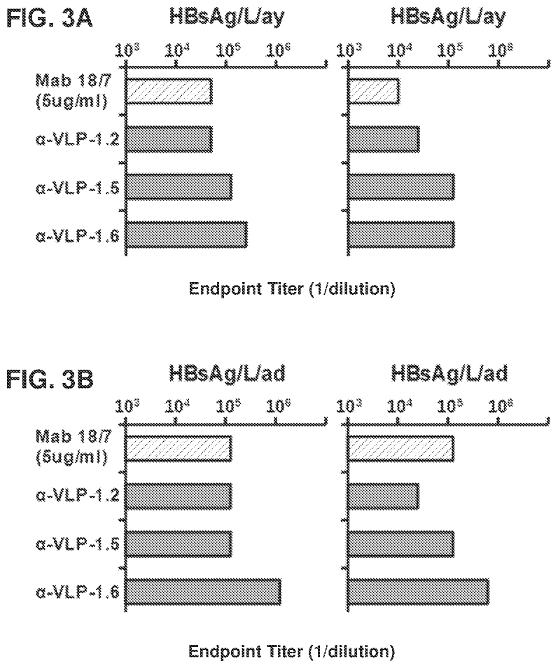

Important characteristics of the PreS1-specific Abs elicited by PreS1-WHc VLPs include the ability to bind native PreS1 epitopes expressed on L/M/S-HBsAg particles and to recognize both major serotypes ay and ad. Although the PreS1 region is highly conserved, especially the receptor binding N-terminal domain, there are genotype-specific differences. Therefore, anti-PreS1-WHc VLP murine sera were tested by ELISA for binding to a panel of solid phase L/M/S-HBsAg particles purified from infected patients, representing the two major serotypes (FIG. 3). Although the amount of PreS1 sequence among the four L/M/S-HBsAg preparations varied, Mab 18/7 served as a reference antibody. Anti-VLPs 1.2, 1.5 and 1.6 Abs each recognized all four L/M/S-HBsAg particles at varying dilutions, indicating a high degree of cross-reactivity for the major ay and ad serotypes, especially because all of the inserted PreS1 sequences were derived from the ay serotype. Although the results indicate it is probably not necessary to insert genotype-specific PreS1 sequences, if required it can be easily accomplished with this technology.

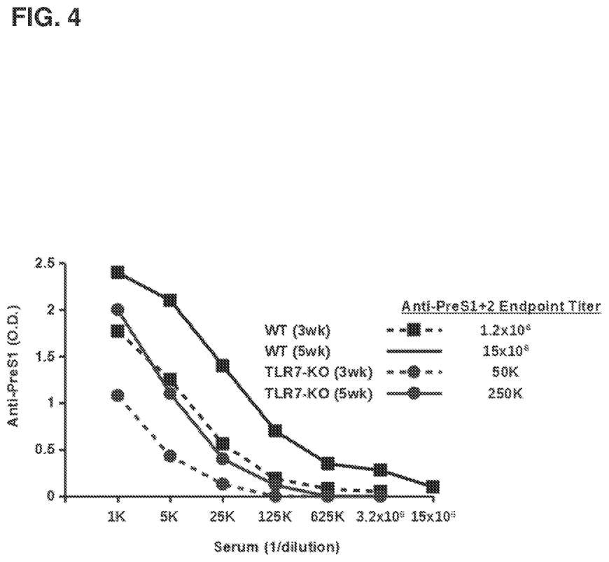

One characteristic of hepadnavirus core proteins that contributes to enhanced immunogenicity is the presence of a domain at the C-terminus that incorporates a TLR7 ligand into the assembled particles (Lee 2009). To determine the influence of TLR7-signaling on the immunogenicity of PreS1-WHc-VLPs, TLR7-KO mice and WT mice were immunized with VLP-1.6 and anti-PreS1 Ab titers were determined three and five weeks after a single injection (FIG. 4). WT mice produced significantly higher endpoint titers of anti-PreS1 Ab at week 3 (1:1.2×10⁶) and at week 5 (1:15×10⁶) as compared to TLR7-KO mice at week 3 (1:50,000) and week 5 (1:250,000). This result indicates that innate TLR7-signaling is operative at least during the primary humoral response to PreS1-WHc VLP immunization.

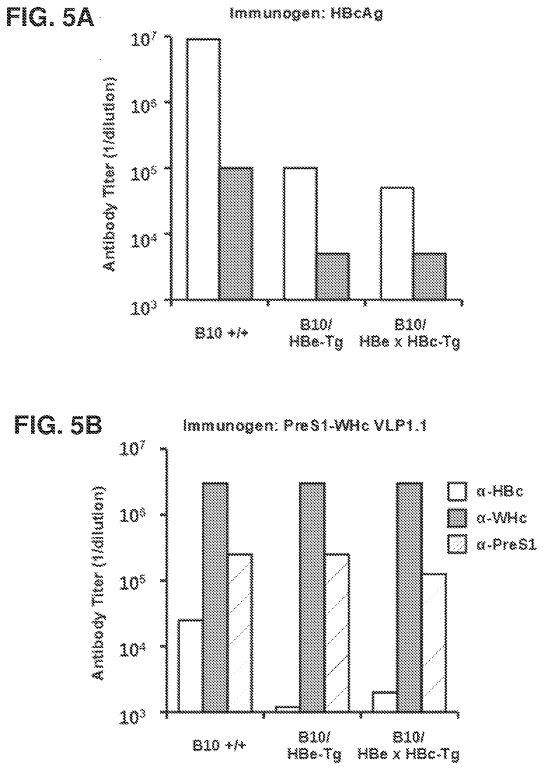

Ability of PreS1-WHc VLPs to circumvent immune tolerance. Several investigators have suggested using the HBcAg as a carrier for PreS protective B cell epitopes in a possible therapeutic vaccine, which is problematic given immune tolerance to the HBc/HBeAgs in chronic hepatitis B virus infection, especially during the immune tolerant phase (Bremer 2011; Chen 2004b). As a means of circumventing immune tolerance, the WHcAg has been utilized as a carrier of 8 PreS1 B cell epitopes. HBeAg-MUP-Tg mice and HBeAg x HBcAg-MUP double-Tg mice are known to be extremely tolerant to the HBc/HBeAgs at the Th/CTL levels (Frelin 2009). Consequently immunization of the Tg mice with 20 μg, HBcAg in IFA resulted in a high degree of Th cell tolerance as reflected by a 900-fold reduction in anti-HBc antibody as compared to WT B10 mice (FIG. 5A) and a 20-fold reduction in cross-reactive anti-WHc Abs. The low level anti-HBc Abs in the HBe/HBcAg-Tg mice reflects the contribution of T cell independent antibody production (Milich 1986b). In contrast, immunization of HBeAg-MUP-Tg or HBe/HBcAg-MUP Tg mice with a PreS1-WHc VLP (VLP-1.1) yielded high titer anti-WHc and anti-PreS1 Abs comparable to WT mice, whereas, cross-reactive anti-HBc Ab production was significantly reduced (25-12.5-fold) (FIG. 5B). Similarly, immunization with PreS1-WHc VLPs 1.1+ and 1.2 elicited high titer anti-PreS1 Abs in HBeAg-MUP-Tg mice and B10 WT mice.

HBV-Tg mice, which express the HBsAg S/M/L envelope antigens, as well as the HBe/HBcAgs, are immune tolerant to the HBV structural antigens (Kakimi 2002). HBV-Tg mice were immunized and boosted with a mixture of 20 μg each of PreS1-WHc VLPs (1.2, 1.3 and 1.6) and anti-PreS1 humoral responses compared to WT mice (Table 4). Anti-PreS1 antibody production in HBV-Tg mice detected by binding to the rPreS1/2 protein, HBV virions and 3 of 5 PreS1 peptides was equivalent to or higher than in WT mice and lower against two peptides (aa18-25 and aa83-106), possibly due to greater adsorption of these anti-PreS1-specific Abs by circulating PreS1 antigen-bearing particles. Similarly, PreS1-Tg mice immunized and boosted with a combination of VLPs 1.6 and 1.9 produced equivalent or higher titer anti-PreS1 Abs as compared to WT mice with the same exception of anti-aa18-25 and anti-aa83-106 Abs, again suggesting these two specificities may be preferentially adsorbed by circulating PreS1 antigen (Table 4). It is important to note that anti-PreS1 Ab production did not result in liver injury in either HBV-Tg or PreS1-Tg mice as determined by the lack of serum ALT elevation.

TABLE 4
Comparative Immunogenicity of PreS1-WHc VLPs in HBV-Tg, PreS1-Tg and
Wild Type Mice

| | | | Antibody Titers (1/dilution) vs. PreS1 Antigens | | | | | |
|---|---|---|---|---|---|---|---|---|
| Mice | n | HBV | rPreS1 + 2 | p(1-15) | p(1-36) | p(18-25) | p(21-42) | p(83-106) |
| Wild Type | 5 | 50K | 125K | 125K | 3 × 10⁶ | 625K | 625 K | 125K |
| (B6/BALB/c) | | | | | | | | |
| HBV-Tg | 5 | 72K | 190K | 625K | 6 × 10⁶ | 50K | 875K | 0.5K |
| (B6/BALB/c) | | | | | | | | |
| PreS1-Tg | 6 | 110K | 2 × 10⁶ | 70K | 3 × 10⁶ | 70K | 900K | 14K |
| (B6) | | | | | | | | |

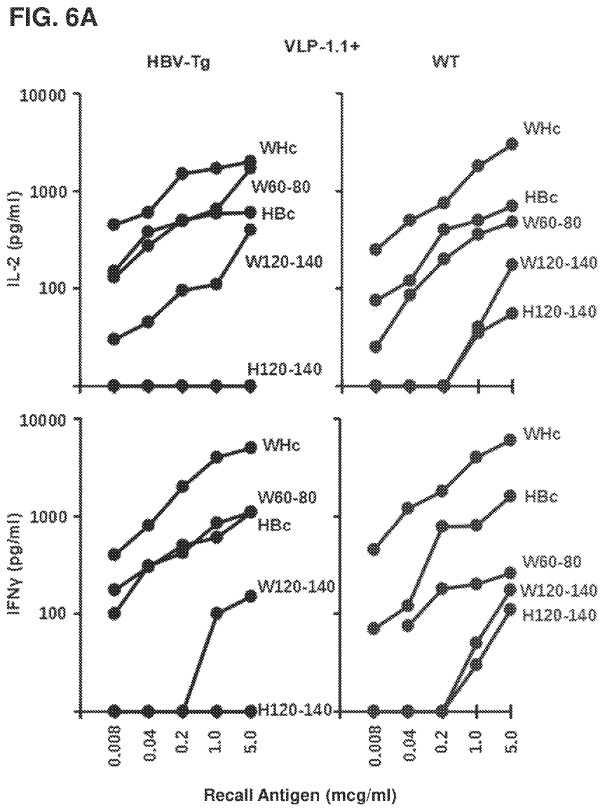

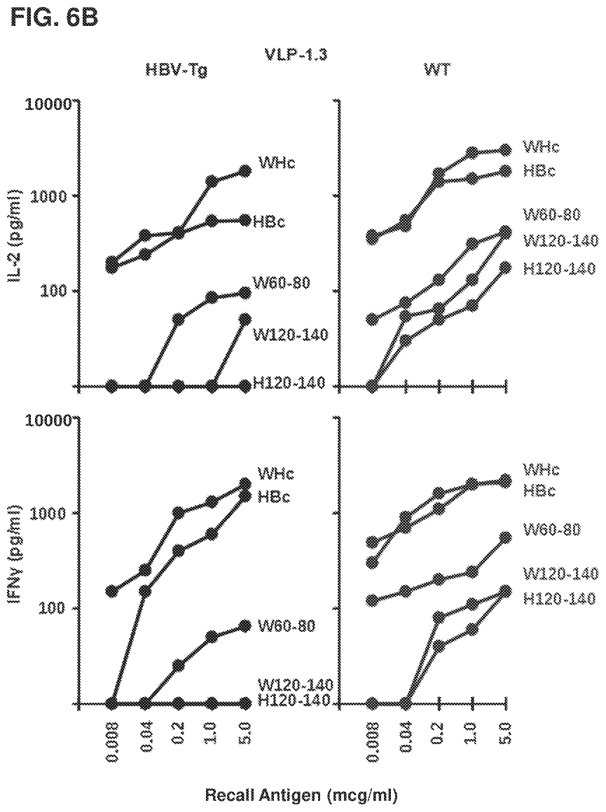

To analyze the Th cell response to PreS1-WHc VLPs, HBV-Tg and WT mice were immunized with either VLP-1.1+ or VLP-1.3 and splenic IL-2 and IFNγ cytokine production in response to culture with a panel of recall antigens was determined (FIG. 6). As expected, native WHcAg was the dominant source of Th cell cytokine production in response to PreS1-WHc VLP immunization, as well as the constituent WHcAg-derived peptides W60-80 and W120-140, in both HBV-Tg and WT mice. Note that Th cells cross-reactive for the HBcAg were also primed by immunization with PreS1-WHc VLPs 1.1+ and 1.3 but produced IL-2 and IFN-γ to a lesser degree than in response to the WHcAg, especially in HBV-Tg mice. However, the fine specificity of the cross-reactive HBcAg-specific T cells could not be determined in HBV-Tg mice because HBV-Tg mice on a B6/BALBc background are tolerant to the H120-140 dominant HBcAg-specific T cell site, unlike WT mice, in which H120-140-specific Th cells were detected. The W120-140 and H120-140 sequences differ by only two amino acids. Therefore, WHcAg-specific Th cells were dominant in PreS1-WHc VLP immunized mice, although low level HBcAg-cross-reactive Th cells were also primed even in HBV-Tg mice. However, the lack of a H120-140-specific Th cell response was evidence of HBcAg-specific Th cell tolerance in HBV-Tg mice.

In summary, the results from immunization studies in HBe/HBcAg-MUP-Tg mice, HBV-Tg mice and PreS1-Tg mice revealed that use of the WHcAg platform to carry PreS1 B cell epitopes was capable of circumventing HBe/HBcAg-specific and L/M/S-HBsAg-specific Th cell tolerance, which characterizes CHB infection. It is also notable that the HBV-Tg and PreS1-Tg lineages were not tolerant at the B cell level to PreS1 B cell epitopes. The presence of PreS1/2-specific, as well as HBsAg-specific IgG in immune complexes in patients with chronic active hepatitis is also consistent with a lack of B cell tolerance during the immune clearance phases of chronic HBV infection (Maruyama 1993).

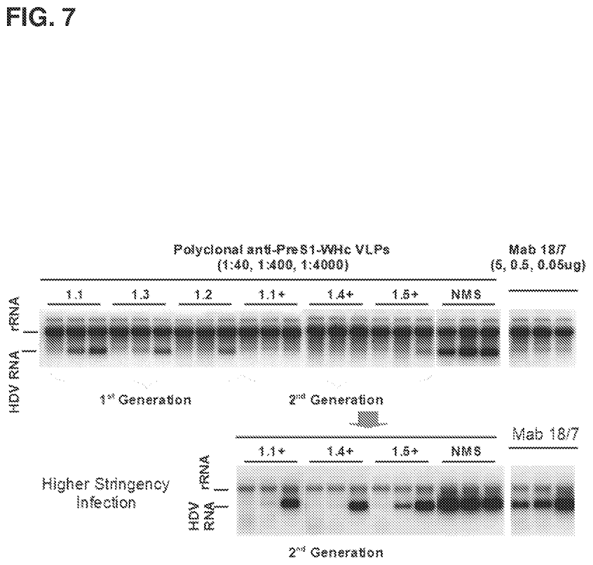

Immunization with PreS1-WHc VLPs elicits HBV neutralizing antibodies. Previous results indicated that PreS1-WHc VLPs were capable of circumventing Th cell immune tolerance and can elicit high titer anti-PreS1-specific Abs of at least 8 different specificities. However, to be relevant, the anti-PreS1-specific Abs produced must be virus-neutralizing (i.e., nAb). As shown in FIG. 7, the anti-PreS1 antibodies elicited by immunization with 6 PreS1-WHc VLPs efficiently neutralized/prevented infection of a HepaRG human hepatocyte cell line by a hepatitis delta virus (HDV) coated with HBV envelope proteins (i.e., L/M/S HBsAg/ay) in an infection assay (Blanchet 2006). Note that antisera to PreS1-WHc VLPs 1.4+ and 1.1+ were capable of completely preventing HDV-HBV infection of HepaRG cells even at final dilutions of 1:4000, as did 0.05 μg/ml of Mab 18/7 (a standard anti-PreS1 neutralizing Mab). In an attempt to find an endpoint dilution for viral neutralization, a higher stringency infection was performed (lower panel, FIG. 7). In the high stringency assay Mab 18/7 even at 5.0 μg/ml was not able to completely neutralize infection. However, the 3 polyclonal anti-PreS1-WHc VLP antisera completely neutralized infection at final dilutions of 1:400 (VLPs 1.1+ and 1.4+) and 1:40 (VLP 1.5). Similarly, anti-PreS1-WHc VLP 1.6 antibody completely prevented infection at a final dilution of 1:400.

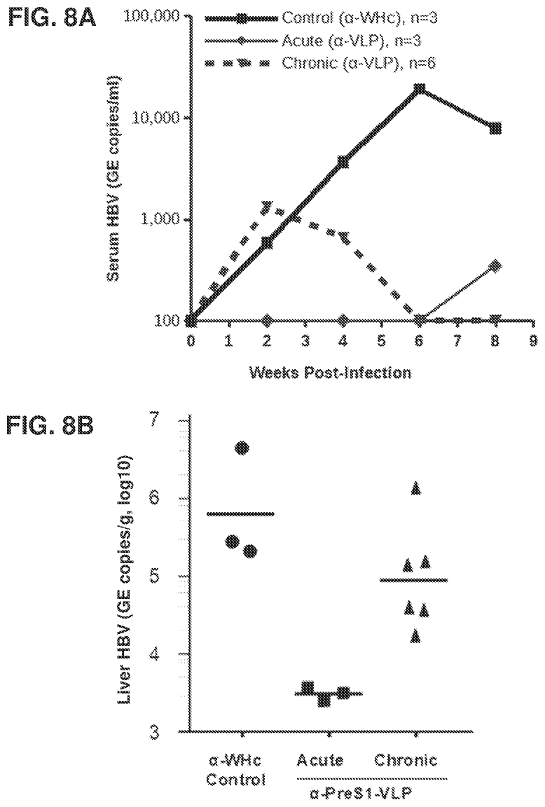

Anti-PreS1-WHc Abs prevent acute infection and clear serum HBV in previously-infected mice in vivo in human-liver chimeric mice. In addition to the ability of PreS1-specific Abs to neutralize HBV infection of a human hepatocyte cell line in vitro (FIG. 7), to determine the efficacy of PreS1-specific nAbs in an infectious in vivo system mice made chimeric with human liver cells were utilized (Bility 2012; Bility 2014). Human-liver chimeric mice are immune compromised, so first WT mice were immunized with a combination of VLP-1.6, VLP-1.2 and VLP-1.3+ and 0.2 ml of secondary anti-PreS1 antisera or control anti-WHc sera was adoptively transferred into human-liver chimeric mice: (1) prior to HBV infection (acute infection and controls); or (2) 2 weeks after HBV infection (“chronic infection”) with 1×10⁶ HBV GE copies/mouse in each challenge. HBV DNA in the serum was monitored every 2 weeks for 8 weeks and HBV DNA in the liver was determined at termination at 8 weeks post-infection (FIG. 8). Control mice demonstrated escalating serum HBV DNA levels that peaked at 6-8 weeks post-infection. The 4 mice adoptively transferred with anti-PreS1 Abs prior (day −1) to HBV infection were protected against acute infection with the exception of one “breakthrough” at 8 weeks post-infection, as nAb levels waned. The acute group only received a single injection of 0.2 ml of anti-PreS1 sera, while the chronic group received adoptive transfer of 0.2 ml of anti-PreS1 sera at 2 and 5 weeks after the HBV infection. All chronically infected mice cleared serum HBV DNA by week 6 post-infection and remained negative for serum HBV DNA at the termination of the experiment (FIG. 8A). At termination, liver HBV DNA levels were determined and no virus was detected in the livers of the acute group. Interestingly, the HBV DNA levels in the livers of the chronic group were approximately 1-log lower than those in the control group (FIG. 8B). Anti-PreS1 nAb were not expected to clear the pre-existing infection in the liver and the reduced HBV DNA liver load compared to controls most likely represented the ability of circulating anti-PreS1 nAbs to prevent viral spreading to uninfected hepatocytes since HBV infection requires secretion of cell-free virus. Consistent with this interpretation, immunohistology staining for HBsAg detected significant HBsAg in the control livers, no HBsAg in the acute livers and minor staining in the “chronic” livers.

These results provide a proof-of-concept that the PreS1-WHc VLPs can elicit nAbs capable of preventing an acute HBV infection into human liver cells. Moreover, the nAbs are sufficient to clear serum HBV from “chronically infected” mice and prevent spreading of HBV amongst human liver cells in vivo.

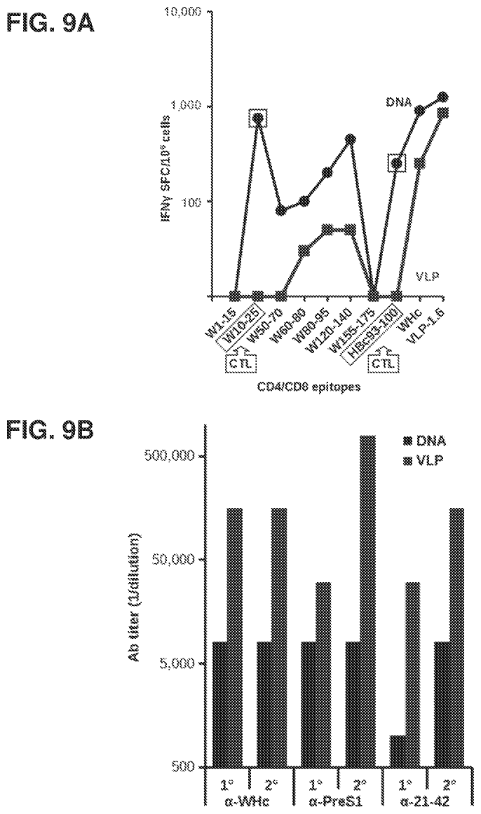

WHcAg-based DNA constructs designed to circumvent immune tolerance and to elicit HBcAg-specific CTL. As described above, it was demonstrated that immunization with PreS1-WHc VLPs could elicit noncross-reactive WHcAg-specific hetero-specific CD4⁺ T cells and to a lesser degree WHcAg/HBcAg cross-reactive CD4⁺ T cells that may mediate viral clearance via cytokine production. For example, immunization with PreS1-WHc VLP 1.1+ primed HBcAg-cross-reactive CD4⁺ T cells in both WT and HBV-Tg mice (FIG. 6). To determine the ability of the PreS1-WHc VLPs to elicit a CD8⁺ CTL response in either a DNA or VLP format, groups of mice were immunized with either VLP-1.6 protein or DNA encoding VLP-1.6. As shown in FIG. 9A, the DNA version elicited superior CD4+ T cell responses to VLP-1.6 and its constituent 4 WHc-specific CD4⁺ T cell epitopes (WHc50-70, WHc60-80, WHc 80-95 and WHc120-140) as compared to the protein version of VLP-1.6. Moreover, the DNA immunogen elicited a strong CD8⁺ CTL response to WHc10-25 and a cross-reactive CTL response to the HBcAg-specific HBc93-100 CTL epitope, whereas, protein VLP-1.6 did not prime any CTL responses. Therefore, just as cross-reactive HBcAg-specific CD4⁺ T cells can be primed by PreS1-WHc VLPs (FIG. 6), cross-reactive HBcAg-specific CD8+ T cells can be primed by DNA encoding PreS1-WHc VLPs (FIG. 9A). It is also notable that, despite the superior priming of WHcAg-specific CD4⁺ T cells by the DNA vaccine compared to the VLP protein, the VLP-1.6 protein immunization elicited far superior (at least 10× higher) anti-WHc and anti-PreS1 Ab responses (FIG. 9B). This was a dramatic demonstration of the complementarity between the two forms of immunization and that the best method to ensure high titer nAb production, as well as effective CTL responses, is to use a VLP prime/DNA boost strategy. These WHcAg/HBcAg cross-reactive CD4⁺/CD8⁺ T cells represented only one method of eliciting HBcAg-specific CD4⁺/CD8⁺ T cells.

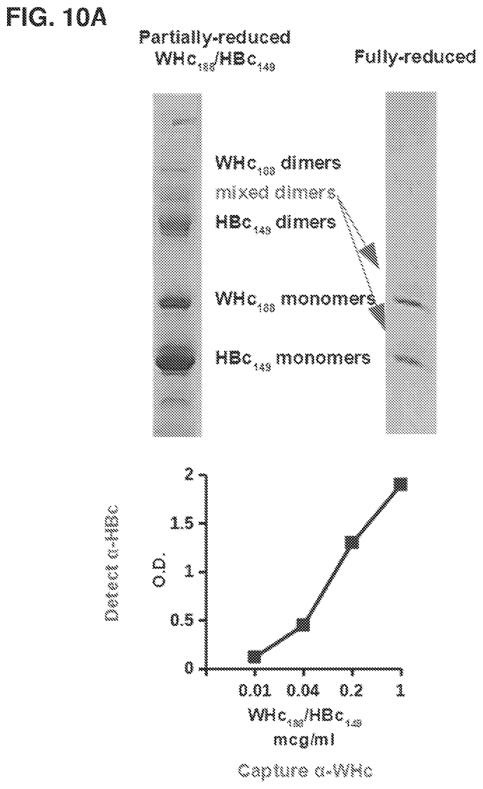

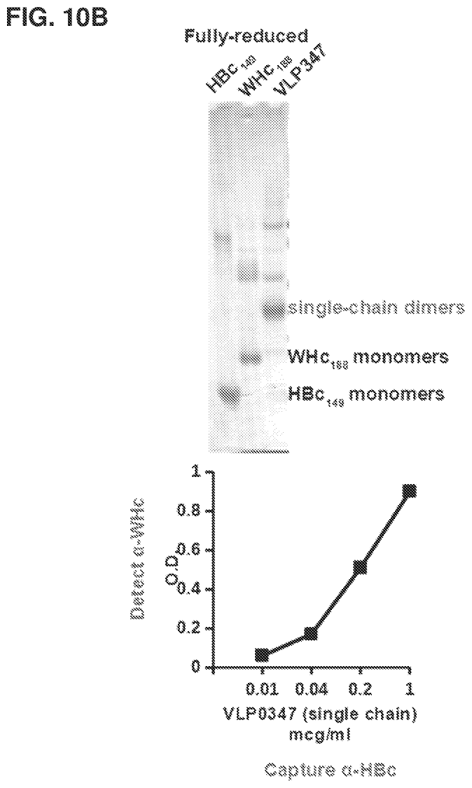

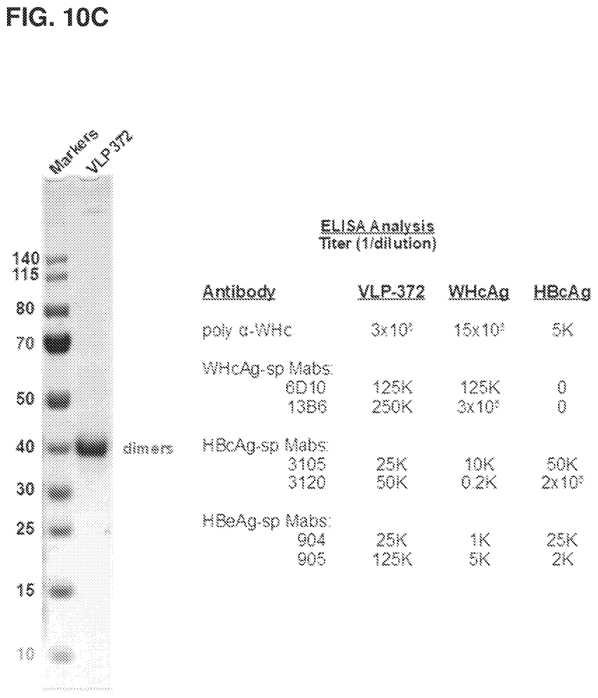

It was anticipated that the WHcAg-“hetero-specific” Th cells primed by a hybrid WHcAg/HBcAg DNA vaccine was contemplated to enable the direct priming of a significantly stronger HBcAg-specific CD8⁺ CTL response. Therefore, the goal was to co-express the WHcAg with the HBcAg in the same VLP in order to allow WHcAg-specific (hetero-specific) Th cells to provide T cell help for the priming and maintenance of HBcAg-specific CD8⁺ CTLs (see FIG. 1). Because the WHcAg and the HBcAg are 68% homologous and structurally very similar it was possible to obtain hybrid WHcAg/HBcAg assembled VLPs in E. coli using two different strategies. First, full-length WHcAg₁₈₈ and truncated HBcAg₁₄₉ genes were co-expressed in E. coli to form hybrid WHcAg/HBcAg VLPs. The subunit for assembly of this VLP is a dimer and biochemical analysis of VLPs from E. coli co-expressing WHcAg and HBcAg showed that, in addition to homodimers, there was a significant fraction of mixed WHcAg/HBcAg dimers, indicating the presence of hybrid WHcAg/HBcAg VLPs (FIG. 10A). The presence of hybrid WHcAg/HBcAg VLPs was confirmed by ELISA analysis utilizing a WHcAg-specific mAb that did not cross-react with HBcAg, and reciprocally an HBcAg-specific mAb that did not cross-react with WHcAg to capture and hence detect hybrid VLPs in solution (FIG. 10, lower panels). The hybrid-specific ELISAs did not detect either WHcAg or HBcAg homogeneous particles.

The second method used was to fuse the HBcAg₁₄₉ gene to the N-terminus of the WHcAg₁₈₈ gene with a dimer linker to form a “single-chain dimer” and to express the one contiguous open reading frame in E. coli. Again, the biochemical and ELISA analysis indicated that hybrid WHcAg/HBcAg VLPs were produced, which were designated as VLP-347 (FIG. 10B), and VLP-372 (FIG. 10C). The advantage of the single polypeptide method is that only hybrid VLPs are possible, whereas, separate expression of the two gene constructs could theoretically produce a mixture of homologous and hybrid assembled VLPs.

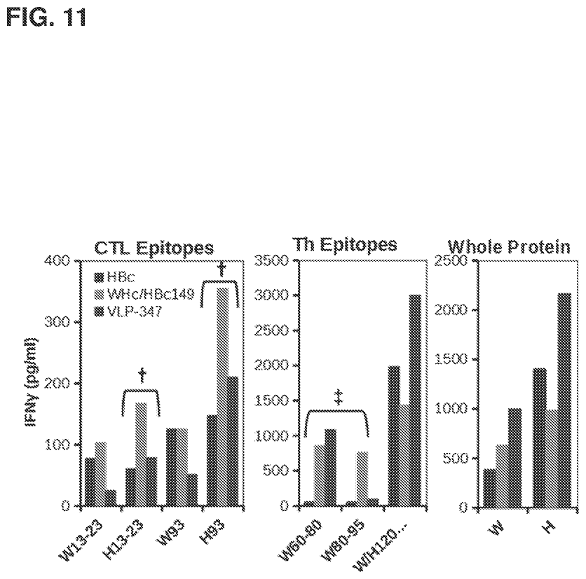

To establish the ability of hybrid WHcAg/HBcAg VLP DNA constructs to elicit HBcAg-specific CTL, B6 mice were immunized intramuscularly (IM) with DNA (100μug, 2 times) encoding VLP-347, hybrid WHcAg₁₈₈/HBcAg149 or HBc alone (FIG. 11). Notably, the hybrid WHcAg/HBcAg DNA construct elicited superior CTL responses as compared to HBcAg-DNA to the dominant CTL epitope on the HBcAg in B6 mice, namely HBc93-100. CTL specific for HBc13-23, presumably due to cross-reactivity with the WHcAg-specific CTL epitope at the WHc13-23 site, were also produced. Because a dominant CD4+ Th cell site in B6 mice (aa120-140) is highly conserved (19 of 21aa) between WHcAg and HBcAg, it was not surprising that the WHcAg and HBcAg 120-140 peptides recalled a strong IFNγ response after immunization with all 3 DNA constructs. However, the H120-140 Th epitope is not functional in HBV-Tg mice due to immune tolerance (see FIG. 6). Note that IFNγ responses to the WHcAg-specific Th epitopes WHc60-80 and WHc80-95 were primed by the VLP-347 and hybrid WHc188/HBc149 DNAs, but not by HBc DNA immunization. Therefore, in HBV-Tg mice these “hetero-specific”, WHcAg-unique T cells are contemplated to be able to replace the defective homo-specific H120-140-specific Th cells and become the dominant source of Th cells for the induction of HBcAg-specific CTL.

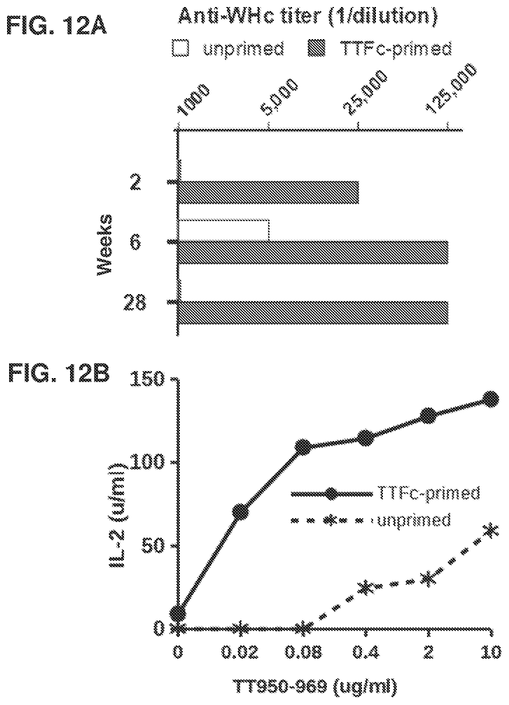

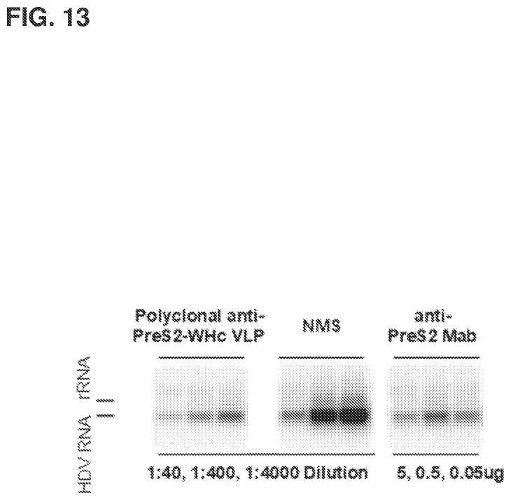

It is also possible to insert so-called “universal” Th cell epitopes derived from tetanus (TT) and diphtheria (DT) toxoid proteins into the PreS1-WHc VLPs and the hybrid WHcAg/HBcAg DNA constructs in order to elicit additional hetero-specific CD4⁺ Th cells. This is most relevant in HBV/HIV co-infection as TT/DT-specific memory Th cells most likely were primed prior to HIV/HBV infections and remain viable alternatives to provide “hetero-specific” Th cell function for HBV-specific B cells and CTL. As shown in FIG. 12, TT-immune mice (a model for T-immunized humans) produced early and enhanced anti-WHc Ab responses when injected with a single dose of WHcAg-TT VLPs via the action of T-hetero-specific Th cells.

### REFERENCES

Aida and Pabst. Removal of endotoxin from protein solutions by phase separation using Triton X-114. J Immunol Methods 1990; 132:191-5.
Alberti et al. Fine specificity of human antibody response to the PreS1 domain of hepatitis B virus. Hepatology 1990; 12:199-203.
Alexander et al. Linear PADRE T helper epitope and carbohydrate B cell epitope conjugates induce specific high titer IgG antibody responses. Journal Immunol 2000; 164: 1625-1633.
Ausubel et al. Current Protocols in Molecular Biology (Greene & Wiley, New York). Ausubel Current Protocols in Molecular Biology 1987.
Barrera et al. Mapping of the hepatitis B virus pre-S1 domain involved in receptor recognition. J Virol 2005; 79:9786-98.
Bility et al. Generation of a humanized mouse model with both human immune system and liver cells to model hepatitis C virus infection and liver immunopathogenesis. Nat Protoc 2012; 7:1608-17.
Bility et al. Hepatitis B virus infection and immunopathogenesis in a humanized mouse model: induction of human-specific liver fibrosis and M2-like macrophages. PLoS Pathog 2014; 10:e1004032.
Billaud et al. Combinatorial approach to hepadnavirus-like particle vaccine design. J Virol 2005; 79:13656-66.
Blanchet and Sureau. Analysis of the cytosolic domains of the hepatitis B virus envelope proteins for their function in viral particle assembly and infectivity. J Virol 2006; 80:11935-45.
Bremer et al. N-terminal myristoylation-dependent masking of neutralizing epitopes in the preS1 attachment site of hepatitis B virus. J Hepatol 2011; 55:29-37.
Chen et al. A function of the hepatitis B virus precore protein is to regulate the immune response to the core antigen. Proc Natl Acad Sci USA 2004a; 101:14913-8.
Chen et al. Recombinant hepatitis B core antigen carrying preS1 epitopes induce immune response against chronic HBV infection. Vaccine 2004b; 22:439-46.
Coligan et at. Current Protocols in Immunology. Greene & Wiley, New York. 1991.
Conway et at. Visualization of a 4-helix bundle in the hepatitis B virus capsid by cryo-electron microscopy. Nature 1997; 386: 91-94.
Dakappagari et at. A chimeric multi-human epidermal growth factor receptor-2 B cell epitope peptide vaccine mediates superior antitumor responses. Journal Immunol 2003; 170: 4242-4253.
Demotz et at. The set of naturally processed peptides displayed by DR molecules is tuned by polymorphism of residue 86. European J Immunol 1993; 23: 425-432.
Diethelm-Okita et at. Universal epitopes for human CD4+ cells on tetanus and diphtheria toxins. J Infect Dis 2000; 181: 1001-1009.
Falugi et al. Rationally designed strings of promiscuous CD4+ T cell epitopes provide help to Haemophilus influenzae type b oligosaccharide: a model for new conjugate vaccines. European J Immunol 2001; 31: 3816-3824.
Fattovich et al. Natural history of chronic hepatitis B: special emphasis on disease progression and prognostic factors. J of Hepatol 2008; 48: 335-352.
Frelin et al. A mechanism to explain the selection of the hepatitis e antigen-negative mutant during chronic hepatitis B virus infection. J Virol 2009; 83:1379-92.
Freshney. Culture of Animal Cells: A Manual of Basic Technique Wiley-Liss & Sons. Inc, New York 1994.
Glebe et al. Mapping of the hepatitis B virus attachment site by use of infection-inhibiting preS1 lipopeptides and tupaia hepatocytes. Gastroenterology 2005; 129:234-45.
Greenstein et al. A universal T cell epitope-containing peptide from hepatitis B surface antigen can enhance antibody specific for HIV gp120. Journal Immunol 1992; 148: 3970-3977.
Guidotti et al. High-level hepatitis B virus replication in transgenic mice. J Virol 1995; 69:6158-69.
Hammer et at. Promiscuous and allele-specific anchors in HLA-DR-binding peptides. Cell 1993; 74: 197-203.
Harlow et at. Antibodies: A Laboratory Manual. New York: Cold Spring Harbor Laboratory 1988.
Heermann et al. Large surface proteins of hepatitis B virus containing the pre-s sequence. J Virol 1984; 52:396-402.
Kakimi et al. Immunogenicity and tolerogenicity of hepatitis B virus structural and nonstructural proteins: implications for immunotherapy of persistent viral infections. J Virol 2002; 76:8609-20.
Lee et al. Interaction of the hepatitis B core antigen and the innate immune system. J Immunol 2009; 182:6670-81.
Li et al. Efficient genetic manipulation of the NOD-Rag1-/-IL2RgammaC-null mouse by combining in vitro fertilization and CRISPR/Cas9 technology. Sci Rep 2014; 4:5290.
Li et al. Studying HBV Infection and Therapy in Immune-Deficient NOD-Rag1-/-IL2RgammaC-null (NRG) Fumarylacetoacetate Hydrolase (Fah) Knockout Mice Transplanted with Human Hepatocytes. Methods Mol Biol Clifton N.J. 2017; 1540:267-76.
Lok. The maze of treatments for hepatitis B. N Engl J Med 2005; 352:2743-6.
Maruyama et al. The serology of chronic hepatitis B infection revisited. J Clin Invest 1993; 91:2586-95.
Michel et al. Therapeutic vaccines and immune-based therapies for the treatment of chronic hepatitis B: perspectives and challenges. J Hepatol 2011; 54:1286-96.
Milich et al. Immune response to the pre-S(1) region of the hepatitis B surface antigen (HBsAg): a pre-S(1)-specific T cell response can bypass nonresponsiveness to the pre-S(2) and S regions of HBsAg. J Immunol 1986a; 137:315-22.
Milich and McLachlan. The nucleocapsid of hepatitis B virus is both a T-cell-independent and a T-cell-dependent antigen. Science 1986b; 234:1398-401.
Milich. The Concept of Immune Tolerance in Chronic Hepatitis B Virus Infection Is Alive and Well. Gastroenterology 2016; 151:801-4.
Mullis et al. PCR. The Polymerase Chain Reaction. Methods in Molecular Biology Series 1994
Nakamoto et al. Immune pathogenesis of hepatocellular carcinoma. J Exp Med 1998; 188:341-50.
Neurath et al. Identification and chemical synthesis of a host cell receptor binding site on hepatitis B virus. Cell 1986; 46:429-36.
Neurath et al. Antibodies to synthetic peptides from the pre-S1 and pre-S2 regions of one subtype of the hepatitis B virus (HBV) envelope protein recognize all HBV subtypes. Mol Immunol 1987; 24:975-80.
Neurath et al. Antibodies to synthetic peptides from the preS1 region of the hepatitis B virus (HBV) envelope (env) protein are virus-neutralizing and protective. Vaccine 1989; 7:234-6.
Protzer and Knolle. “To Be or Not to Be”: Immune Tolerance in Chronic Hepatitis B. Gastroenterology 2016; 151:805-6.
Sambrook et al. Molecular cloning: a laboratory manual. No. Ed. 2. Cold spring harbor laboratory press, 1989.
Schödel et al. The position of heterologous epitopes inserted in hepatitis B virus core particles determines their immunogenicity. J Virology 1992; 66: 106-114.
Schödel et al. Immunity to malaria elicited by hybrid hepatitis B virus core particles carrying circumsporozoite protein epitopes. J of Exp Med 1994; 180: 1037-1046.
Thornton et al. Protection of chimpanzees from hepatitis-B virus infection after immunization with synthetic peptides: identification of protective epitopes in the pre-S region. In: Lerner R, et al. editors. Modern Approaches to New Vaccines Including Prevention of AIDS. Cold Spring Harbor Laboratory Publications; 1989. page 467-471.

SEQUENCES

SEQ ID NO: 1
Woodchuck hepatitis virus
>WHcAg full length (X = C or S)
MDIDPYKEFGSSYQLLNFLPLDFFPDLNALVDTATALYEEELTGREHCSPHHTAIRQALVXWDELTKLIA
WMSSNITSEQVRTIIVNHVNDTWGLKVRQSLWFHLSCLTFGQHTVQEFLVSFGVWIRTPAPYRPPNAPIL
STLPEHTVIRRRGGARASRSPRRRTPSPRRRRSQSPRRRRSQSPSANC
SEQ ID NO: 2
Woodchuck hepatitis virus
>WHcAg truncated (X = C or S)
MDIDPYKEFGSSYQLLNFLPLDFFPDLNALVDTATALYEEELTGREHCSPHHTAIROALVXWDELTKLIA
WMSSNITSEQVRTIIVNHVNDTWGLKVRQSLWFHLSCLTFGQHTVQEFLVSFGVWIRTPAPYRPPNAPIL
STLPEHTVI
SEQ ID NO: 3
Human hepatitis B virus
>HBcAg full length ( X = C or S)
MDIDPYKEFGATVELLSFLPSDFFPSVRDLLDTASALYREALESPEHCSPHHTALRQAILXWGELMTLAT
WVGVNLEDPASRDLVVSYVNTNMGLKFRQLLWFHISCLTFGRETVIEYLVSFGVWIRTPPAYRPPNAPIL
STLPETTVVRRRGRSPRRRTPSPRRRRSQSPRRRRSQSRESQC
SEQ ID NO: 4
Human hepatitis B virus
>HBcAg truncated (X = C or S)
MDIDPYKEFGATVELLSFLPSDFFPSVRDLLDTASALYREALESPEHCSPHHTALRQAILXWGELMTLAT
WVGVNLEDPASRDLVVSYVNTNMGLKFRQLLWFHISCLTFGRETVIEYLVSFGVWIRTPPAYRPPNAPIL
STLPETTVV
SEQ ID NO: 5
Consensus
>full length (X is any amino acid)
MDIDPYKEFGXXXXLLXFLPXDFFPXXXXLXDTAXALYXEXLXXXEHCSPHHTAXRQAXXXWXELXXLXX
WXXXNXXXXXXRXXXVXXVNXXXGLKXRQXLWFHXSCLTFGXXTVXEXLVSFGVWIRTPXXYRPPNAPIL
STLPEXTVXRRRGXXXXXRSPRRRTPSPRRRRSQSPRRRRSQSXXXXC
SEQ ID NO: 6
Consensus
truncated (X is any amino acid)
MDIDPYKEFGXXXXLLXFLPXDFFPXXXXLXDTAXALYXEXLXXXEHCSPHHTAXRQAXXXWXELXXLXX
WXXXNXXXXXXRXXXVXXVNXXXGLKXRQXLWFHXSCLTFGXXTVXEXLVSFGVWIRTPXXYRPPNAPIL
STLPEXTVX
SEQ ID NO: 7
>Hepatitis B Virus PreS1 (ayw)
MGQNLSTSNP LGFFPDHQLD PAFRANTANP DWDFNPNKDT WPDANKVGAG AFGLGFTPPH
GGLLGWSPQA QGILQTLPAN PPPASTNRQS GRQPTPLSPP LRNTHPQA
SEQ ID NO: 8
>Hepatitis B Virus PreS2 (ayw)
MQ WNSTTFHQTL QDPRVRGLYF PAGGSSSCTV NPVLTTASPL SSIFSRIGDP ALN
SEQ ID NO: 9
>Hepatitis B Virus PreS1 + S2 (ayw)
MGQNLSTSNP LGFFPDHQLD PAFRANTANP DWDFNPNKDT WPDANKVGAG AFGLGFTPPH
GGLLGWSPQA QGILQTLPAN PPPASTNRQS GRQPTPLSPP LRNTHPQAMQ WNSTTFHQTL
QDPRVRGLYF PAGGSSSGTV NPVLTTASPL SSIFSRIGDP ALN
SEQ ID NO: 10
>Hepatitis B Virus Small Surface Antigen
MENITSG FLGPLLVLQA
GFFLLTRILT IPQSLDSWWT SLNFLGGTTV CLGQNSQSPT SNHSPTSCPP TCPGYRWMCL
RRFIIFLFIL LLCLIFLLVL LDYQGMLPVC PLIPGSSTTS TGPCRTCMTT AQGTSMYPSC
CCTKPSDGNC TCIPIPSSWA FGKFLWEWAS ARFSWLSLLV PFVQWFVGLS PTVWLSVIWM
MWYWGPSLYS ILSPFLPLLP IFFCLWVYI
SEQ ID NO: 11
>Hepatitis B Virus Medium Surface Antigen
MQ WNSTTFHQTL QDPRVRGLYF PAGGSSSGTV NPVLTTASPL SSIFSRIGDP ALN
MENITSG FLGPLLVLQA
GFFLLTRILT IPQSLDSWWT SLNFLGGTTV CLGQNSQSPT SNHSPTSCPP TCPGYRWMCL
RRFIIFLFIL LLCLIFLLVL LDYQGMLPVC PLIPGSSTTS TGPCRTCMTT AQGTSMYPSC
CCTKPSDGNC TCIPIPSSWA FGKFLWEWAS ARFSWLSLLV PFVQWFVGLS PTVWLSVIWM
MWYWGPSLYS ILSPFLPLLP IFFCLWVYI
SEQ ID NO: 12
>Hepatitis B Virus Large Surface Antigen

| | |
|---|---|
| MGQNLSTSNP LGFFPDHQLD PAFRANTANP DWDFNPNKDT WPDANKVGAG AFGLGFTPPH | 61 |
| GGLLGWSPQA QGILQTLPAN PPPASTNRQS GRQPTPLSPP LRNTHPQAMQ WNSTTFHQTL | 121 |
| QDPRVRGLYF PAGGSSSGTV NPVLTTASPL SSIFSRIGDP ALNMENITSG FLGPLLVLQA | 181 |
| GFFLLTRILT IPQSLDSWWT SLNFLGGTTV CLGQNSQSPT SNHSPTSCPP TCPGYRWMCL | 241 |
| RRFIIFLFIL LLCLIFLLVL LDYQGMLPVC PLIPGSSTTS TGPCRTCMTT AQGTSMYPSC | 301 |
| CCTKPSDGNC TCIPIPSSWA FGKFLWEWAS ARFSWLSLLV PFVQWFVGLS PTVWLSVIWM | 361 |
| MWYWGPSLYS ILSPFLPLLP IFFCLWVYI | |

SEQ ID NOs: 13-25 see Table 1
SEQ ID NO: 26
>TT residues 950-969
NNFTVSFWLRVPKVSASHLE
SEQ ID NO: 27
>Primer 1 HBV2270F
5′-GAGTGTGGATTCGCACTCC-3′
SEQ ID NO: 28
>Primer 2 HBV2392R
5′-GAGGCGAGGGAGTTCTTCT-3
SEQ ID NO: 29
internal linker/insert combination
GIL(E)y-Xn-(E)zL
where X is any amino acid, and
n is 50 or less, y is 0, 1 or 2, z is 0, 1 or 2
SEQ ID NO: 30
intervening sequence
EEEE
SEQ ID NO: 31
>intervening sequence
GGGG
SEQ ID NO: 32
>VLP-1.2 aka VRI010b (FLw2-HBV-PreS1.2-78)
MDIDPYKEFGSSYQLLNFLPLDFFPDLNALVDTATALYEEELTGREHCSPHHTAIRQALVCWDELTKLIA
WMSSNITSGILEEPASTNRQSGRQPTPLSPPLRNTHPEELEQVRTIIVNHVNDTWGLKVRQSLWFHLSCL
TFGQHTVQEFLVSFGVWIRTPAPYRPPNAPILSTLPEHTVIRRRGGARASRSPRRRTPSPRRRRSQSPRR
RRSQSPSANC
SEQ ID NO: 33
>VLP-1.3 aka VRI034 (FLw-HBV-PreS1.3-78)
MDIDPYKEFGSSYQLLNFLPLDFFPDLNALVDTATALYEEELTGREHCSPHHTAIRQALVCWDELTKLIA
WMSSNITSGILMGQNLSTSNPLGFFPDHQLDPLEQVRTIIVNHVNDTWGLKVRQSLWFHLSCLTFGQHTV
QEFLVSFGVWIRTPAPYRPPNAPILSTLPEHTVIRRRGGARASRSPRRRTPSPRRRRSQSPRRRRSQSPS
ANC
SEQ ID NO: 34
>VLP-1.6 aka VLP101 (FLw2-HBV-PreS1.6-78/79)
MDIDPYKEFGSSYQLLNFLPLDFFPDLNALVDTATALYEEELTGREHCSPHHTAIRQALVCWDELTKLIA
WMSSNITSPDHQLDPAFRANTANPDWDFNPNKDTEQVRTIIVNHVNDTWGLKVRQSLWFHLSCLTFGQHT
VQEFLVSFGVWIRTPAPYRPPNAPILSTLPEHTVIRRRGGARASRSPRRRTPSPRRRRSQSPRRRRSQSP
SANC
SEQ ID NO: 35
>VLP-1.9 aka VLP158 (FLw2(C61S)-HBV-(NtA-PreS1.2)-PreS1.3(+)E-78)
MGPASTNRQSGRQPTPLSPPLRNTHPWLWGAMDIDPYKEFGSSYQLLNFLPLDFFPDLNALVDTATALYE
EELTGREHCSPHHTAIRQALVSWDELTKLIAWMSSNITSGILEMGQNLSTSNPLGFFPDHQLDPAFRAEL
EQVRTIIVNHVNDTWGLKVRQSLWFHLSCLTFGQHTVQEFLVSFGVWIRTPAPYRPPNAPILSTLPEHTV
IRRRGGARASRSPRRRTPSPRRRRSQSPRRRRSQSPSANC
SEQ ID NO: 36
>VLP347 sequence

| | |
|---|---|
| MDIDPNKEFGATVELLSFLPSDFFPSVRDLLDTASALYREALESPEHCSPHHTALRQAIL | 60 |
| CWGELMTLATWVGVNLEDPASRDLVVSYVNTNMGLKFRQLLWFHISCLTFGRETVIEYLV | 120 |
| SFGVWIRTPPAYRPPNAPILSTLPETTVVCGGSGMDIDPYKEFGSSYOLLNFLPLDFFPD | 180 |
| LNALVDTATALYEEELTGREHCSPHHTAIRQALVCWDELTKLIAWMSSNITSEQVRTIIV | 240 |
| NHVNDTWGLKVRQSLWFHLSCLTFGQHTVQEFLVSFGVWIRTPAPYRPPNAPILSTLPEH | 300 |
| TVIRRRGGARASRSPRRRTPSPRRRRSQSPRRRRSQSPSANC | 342 |

SEQ ID NO: 37
>VLP372 sequence

| | |
|---|---|
| MDIDPNKEFGATVELLSFLPSDFFPSVRDLLDTASALYREALESPEHCSPHHTALRQAIL | 60 |
| CWGELMTLATWVGVNLEDPASRDLVVSYVNTNMGLKFRQLLWFHISCLTFGRETVIEYLV | 120 |
| SFGVWIRTPPAYRPPNAPILSTLPETTVVRRRGGARASMDIDPYKEFGSSYQLLNFLPLD | 180 |
| FFPDLNALVDTATALYEEELTGREHCSPHHTAIRQALVCWDELTKLIAWMSSNITSEQVR | 240 |
| TIIVNHVNDTWGLKVRQSLWFHLSCLTFGQHTVQEFLVSFGVWIRTPAPYRPPNAPILST | 300 |
| LPEHTVIRRRGGARASRSPRRRTPSPRRRRSQSPRRRRSQSPSANC | 346 |

SEQ ID NO: 38
>dimer linker
CGGSG
SEQ ID NO: 39
>dimer linker
RRRGGARAS
SEQ ID NO: 40
>UTC
DIEYLNKIQNSLSTEWSPCSVT
SEQ ID NO: 41
>Hepatitis B Virus PreS1 (adr)
MGGWSSKPRQGMGTNLSVPNPLGFFPDHQLDPAFGANSNNPDWDFNPNKDHWPEAIKVGAGDFGPGFTPP
HGGLLGWSPQAQGILTTVPAAPPPVSTNRQSGRQPTPISPPLRDSHPQA
SEQ ID NO: 42
>Hepatitis B Virus PreS2 (adr)
MQWNSTTFHQALLDPRVRGLYFPAGGSSSGTVNPVPTTVSPISSIFSRTGDPAPN
SEQ ID NO: 43
>Hepatitis B Virus PreS1 + S2 (adr)
MGGWSSKPRQGMGTNLSVPNPLGFFPDHQLDPAFGANSNNPDWDFNPNKDHWPEAIKVGAGDFGPGFTPP
HGGLLGWSPQAQGILTTVPAAPPPVSTNRQSGRQPTPISPPLRDSHPQAMQWNSTTFHQALLDPRVRGLY
FPAGGSSSGTVNPVPTTVSPISSIFSRTGDPAPN
SEQ ID NO: 44
> VRI010c aka FLw-HBV-PreS2-74x3-UTC

| | |
|---|---|
| MDIDPYKEFGSSYQLLNFLPLDFFPDLNALVDTATALYEEELTGREHCSPHHTAIRQALV | 60 |
| CWDELTKLIAWMSSEDPRVRGLYFPAGGSSEPSSEDPRVRGLYFPAGGSSEPSSEDPRVR | 120 |
| GLYFPAGGSSEPSSNITSEQVRTIIVNHVNDTWGLKVRQSLWFHLSCLTFGQHTVQEFLV | 180 |
| SFGVWIRTPAPYRPPNAPILSTLPEHTVIRRRGGARASRSPRRGTPSPRRRRSQSPRRRR | 300 |
| SQSPSANCDIEYLNKIQNSLSTEWSPCSVT | 330 |

SEQ ID NO: 45
> PreS2-74x3
EDPRVRGLYFPAGGSSEPSSEDPRVRGLYFPAGGSSEPSSEDPRVRGLYFPAGGSSEPSS

We claim:

1. An antigenic composition comprising a hybrid hepadnavirus core antigen, wherein
the hybrid core antigen is a fusion protein comprising a first portion of a human hepatitis B virus surface antigen (HBsAg) and a woodchuck hepadnavirus core antigen (WHcAg),
the first portion of the HBsAg consists of from 8 to 50 amino acids of the PreS1 domain of the human hepatitis B virus (HBV) large surface antigen,
the amino acid sequence of the WHcAg is at least 95% identical to SEQ ID NO:1,
the amino acid sequence of the PreS1 domain is at least 95% identical to SEQ ID NO:7 or SEQ ID NO:41,
the first portion of the HBsAg is inserted at a first position,
the first position is N-terminus or an internal position of the WHcAg selected from the group consisting of 61, 71, 72, 73, 74, 75, 76, 77, 78, 81, 82, 83, 84, 85 and 92 as numbered according to SEQ ID NO:1, and
the fusion protein is capable of assembling as a hybrid PreS1-WHcAg virus-like particle (VLP).

2. The antigenic composition of claim 1, wherein the first position is an internal position of the core antigen selected from the group consisting of 61, 71, 72, 73, 74, 75, 76, 77, 78, 81, 82, 83, 84, 85 and 92 as numbered according to SEQ ID NO:1, optionally wherein the first position is position 78.

3. The antigenic composition of claim 1, wherein the hybrid core antigen further comprises a second portion of the HBsAg consisting of from 8 to 50 amino acids in length of the PreS1 domain of the large surface antigen, the second portion is inserted at a second position, and the second position is N-terminus or an internal position of the WHcAg selected from the group consisting of 61, 71, 72, 73, 74, 75, 76, 77, 78, 81, 82, 83, 84, 85 and 92 as numbered according to SEQ ID NO:1.

4. The antigenic composition of claim 3, wherein the amino acid sequence of the second portion of the HBsAg is different than the amino acid sequence of the first portion of the HBsAg.

5. The antigenic composition of claim 3, wherein the second position is the N-terminus.

6. The antigenic composition of claim 3, wherein the first position is 78 and the second position is the N-terminus.

7. The antigenic composition of claim 3, wherein the first position is adjacent to the second position, and the first portion and the second portion together are no more than 50 amino acids in length.

8. The antigenic composition of claim 7, wherein the first portion is inserted at position 78 and the second portion is inserted at the C-terminus of the first portion or at the C-terminus of intervening sequence separating the first portion from the second portion, optionally wherein the intervening sequence comprises GGGG (SEQ ID NO:31) or EEEE (SEQ ID NO:30).

9. The antigenic composition of claim 1, wherein the first portion is inserted at an internal site as a linker/insert combination according to the formula GIL(E)y-Xn-(E)zL (SEQ ID NO:29, in which both y and z are in integers independently selected from the group consisting of 0, 1, and 2, and wherein Xn is the first portion.

10. The antigenic composition of claim 1, wherein the WHcAg has a serine at position 61.

11. The antigenic composition of claim 1, wherein the WHcAg as a cysteine at position 61.

12. The antigenic composition of claim 1, wherein the amino acid sequence of:
i) one or both of the first portion and the second portion each comprise one of the group consisting of SEQ ID NOs:13-24; or
ii) one or both of the first portion and the second portion are each at least 95% identical one of the group consisting of SEQ ID NOs:13-24; or
iii) the first portion is selected from the group consisting of SEQ ID NO:15, SEQ ID NO:16, SEQ ID NO:17, and SEQ ID NO:21; or
iv) the second portion is selected from the group consisting of SEQ ID NO:15 and SEQ ID NO:17.

13. The antigenic composition of claim 1, wherein the amino acid sequence of the hybrid PreS1-WHcAg VLP is at least 95% identical to one of the group consisting of SEQ ID NO:32, SEQ ID NO:33, SEQ ID NO:34 and SEQ ID NO:35.

14. The antigenic composition of claim 1, wherein the hybrid PreS1-WHcAg VLP elicits an antibody response against one or more of HBV virions, HBsAg particles, a PreS1 protein consisting of the amino acid sequence of SEQ ID NO:7, and a PreS1+S2 protein consisting of the amino acid sequence of SEQ ID NO:9.

15. The antigenic composition of claim 1, wherein the hybrid PreS1-WHcAg VLP elicits a measurable neutralizing antibody response against HBV.

16. An antigenic composition comprising a hybrid hepadnavirus core antigen, wherein
the hybrid core antigen is a fusion protein comprising a human hepatitis B virus core antigen (HBcAg) and a woodchuck hepadnavirus core antigen (WHcAg), and
the fusion protein is capable of assembling as a hybrid HBcAg-WHcAg virus-like particle (VLP).

17. The antigenic composition of claim 16, wherein the amino acid sequence of the HBcAg is at least 95% identical to SEQ ID NO:4, and the amino acid sequence of the WHcAg is at least 95% identical to SEQ ID NO:1.

18. The antigenic composition of claim 16, wherein a dimer linker of from 5-15 amino acids in length is inserted between the amino acid sequence of the HBcAg and the amino acid sequence of the WHcAg, optionally wherein the dimer linker comprises the amino acid sequence of SEQ ID NO:38 or SEQ ID NO:39.

19. The antigenic composition of claim 16, wherein the amino acid sequence of the hybrid HBcAg-WHcAg virus-like particle (VLP) is at least 95% identical to SEQ ID NO:36 or SEQ ID NO:37.

20. A vaccine comprising the antigenic composition of any one of claims 1-19, and an adjuvant.

21. A polynucleotide encoding the hybrid hepadnavirus core antigen of any one of claims 1-15.

22. An expression construct comprising the polynucleotide of claim 21 in operable combination with a promoter, wherein the promoter drives expression of the hybrid hepadnavirus core antigen in bacterial cells.

23. An expression vector comprising the expression construct of claim 22.

24. A polynucleotide encoding the hybrid hepadnavirus core antigen of any one of claims 16-19.

25. An expression construct comprising the polynucleotide of claim 24 in operable combination with a promoter, wherein the promoter drives expression of the hybrid hepadnavirus core antigen in mammalian cells.

26. An expression vector comprising the expression construct of claim 25.

27. A host cell comprising the expression vector of claim 23 or claim 26, optionally wherein the nucleic acid sequence of the expression construct is optimized for expression in bacterial cells or mammalian cells.

28. A method for eliciting or enhancing an HBsAg-reactive antibody response, the method comprising:
administering to a mammalian subject an effective amount of a vaccine comprising an adjuvant and the antigenic composition of any one of claims 1-15.

29. The method of claim 28, wherein the HBsAg-reactive antibody response comprises antibodies reactive with one or more of HBV virions, HBsAg particles, a PreS1 protein consisting of the amino acid sequence of SEQ ID NO:7, and a PreS1+S2 protein consisting of the amino acid sequence of SEQ ID NO:9.

30. A method for eliciting or enhancing a HBcAg-reactive T lymphocyte response, the method comprising:
administering to a mammal subject an effective amount of the expression vector of claim 25.

31. The method of claim 30, wherein the HBcAg-reactive T lymphocyte response comprises:
i) interferon-gamma secretion inducible by presentation of HBcAg-derived peptides by antigen presenting cells of the mammalian subject; and
ii) HBcAg-specific cytotoxic T lymphocytes.

32. A method for eliciting or enhancing an HBsAg-reactive antibody response and a HBcAg-reactive T lymphocyte response, the method comprising administering to a mammalian subject:
an effective amount of a vaccine comprising an adjuvant and the antigenic composition of any one of claims 1-15; and
an effective amount of the expression vector of claim 25.

33. The method of claim 32, wherein the vaccine and the expression vector are administered concurrently or an separate occasions.

34. The method of claim 33, wherein the vaccine and the expression vector are each administered on 1, 2 or 3 occasions.

35. The method of claim 34, wherein the vaccine and the expression vector are each administered at 1, 2, 3, 4, 5 or 6 month intervals, optionally at 1 or 2 month intervals.

36. The method of claim 33, wherein the vaccine is administered intramuscularly, intradermally or subcutaneously, and the expression vector is administered intramuscularly.

37. The method of claim 28 or claim 29, or any one of claims 32-36, wherein the antigenic composition comprising a plurality hybrid PreS1-WHcAg VLPs, wherein the plurality comprises 2, 3, or 4 different hybrid PreS1-WHcAg VLPs.

38. The method of claim 37, wherein the amino acid sequences of the 2, 3, or 4 different hybrid PreS1-WHcAg VLPs are each at least 95% identical to one of the group consisting of SEQ ID NO:32, SEQ ID NO:33, SEQ ID NO:34 and SEQ ID NO:35.

39. The method of any one of claims 28-38, wherein the mammalian subject is chronically-infected with HBV.

40. The method of claim 39, wherein the mammalian subject is HBeAg-positive.

41. The method of any one of claims 28-38, wherein the mammalian subject is a low or non-responder to a preventative vaccine comprising HBsAg and an aluminum salt.

42. The method of any one of claims 28-38, wherein the mammalian subject is a pregnant HBV-positive carrier.
